SEQUENCE LISTING (4 sequences)

SEQ ID NO: 1 - protein, 7 aa, Artificial Sequence
  Fibrin binding moiety
CDYYGTC

SEQ ID NO: 2 - protein, 6 aa, Artificial Sequence
  Fibrin binding moiety
CPYXLC

SEQ ID NO: 3 - protein, 6 aa, Artificial Sequence
  Fibrin binding moiety
CXYXLC

SEQ ID NO: 4 - protein, 11 aa, Artificial Sequence
  Fibrin binding moiety
FHCXYDLCHIL

(19) United States
(12) Patent Application Publication
(10) Pub. No.: US 2006/0148683 A1
(43) Pub. Date: Jul. 6, 2006

(54) Detection and treatment of intravascular lesions

(76) Inventors: Thomas J. McMurry, Winchester, MA (US); Robert M Weisskoff, Lexington, MA (US)

Correspondence Address: FISH & RICHARDSON P.C., PO BOX 1022, MINNEAPOLIS, MN, 55440-1022, US

(21) Appl. No.: 10/492,108

(22) PCT Filed: Oct. 16, 2002
(86) PCT No.: PCT/US02/33340

§ 371 Date: Sep. 19, 2005

Related U.S. Application Data
(60) Provisional application No. 60/330,156, filed on Oct. 16, 2001.

(51) Int. Cl. A61K 38/12; A61K 38/10

(52) U.S. Cl. 514/9; 514/16; 514/17

(57) ABSTRACT

Optical agents that contain a fibrin binding moiety covalently linked to an optical dye are described, as well as methods of treating intravascular lesions in a patient using such optical agents.

### TECHNICAL FIELD

This invention relates to compositions and methods for the detection and treatment of intravascular lesions, and more particularly to the use of optical agents in conjunction with medical devices to treat intravascular lesions.

### BACKGROUND

Cardiovascular disease is a primary health threat in the developed world. Certain intravascular lesions, such as deep vein thrombosis, pulmonary embolism, and atherosclerotic plaques, are clinical manifestations of cardiovascular disease that have significant morbidity and mortality profiles. For example, in the United States alone, there are an estimated 600,000 patients that suffer pulmonary embolism each year. Approximately 114,000 of these patients later die due to complications associated with the disease.

The high mortality rate is partly due to significant limitations associated with currently available methods to detect intravascular lesions. In particular, identification of intravascular lesions is complicated because of their very location in blood vessels. Blood is a flowing, non-transparent mixture of protein and cells, the net effect of which is a significant background that interferes with detection. As a result, many methods to detect intravascular lesions are inconclusive. In addition, many methods for detection require a time frame that functionally prevents the administration of a treatment in a clinically effective time period.

It would be useful to have a method to treat intravascular lesions that combines sensitive detection of the lesions with immediate access to a therapy designed to reduce the size or to alter the shape of the lesion.

### SUMMARY

This invention relates to compositions and methods for the detection and treatment of intravascular lesions, and more particularly to the use of optical agents in conjunction with medical devices to treat intravascular lesions. The use of the methods and compositions of the present invention enhances the sensitivity and facilitates administration of therapies in a timely fashion. In addition, the methods allow real-time monitoring of the therapy to determine a clinically effective endpoint at which to stop the therapy.

Accordingly, one aspect of the invention provides a method for treating an intravascular lesion in a patient. The term “intravascular lesion” means a lesion within a blood vessel. For example, the lesion can be a thrombus, a clot, an atherosclerotic plaque, or an embolus. The lesion may include fibrin that is exposed to blood flowing in the blood vessel. The method includes administering an optical agent (e.g., orally or parenterally such as intravenously, intraarterially, interstitially, intrathecally, subcutaneously, or intracavity), wherein the optical agent includes a fibrin binding moiety and an optical dye, and wherein the optical agent can form a fibrin-optical agent complex at the site of the lesion. A signal from the fibrin-optical agent complex is detected using a device inserted near the lesion and data is obtained about the lesion based on the signal of the fibrin-optical agent complex. A therapy is then delivered, based on the obtained data, to at least a portion of the lesion, e.g., so that the size of the lesion is reduced or the shape of the lesion is altered.

The fibrin binding moiety may include a peptide. For example, the fibrin binding moiety may include the amino acid sequence Cys-Asp-Tyr-Tyr-Gly-Thr-Cys (SEQ ID NO: 1), the amino acid sequence Cys-Pro-Tyr-Xaa-Leu-Cys (SEQ ID NO:2), where Xaa can be Gly or Asp, or the amino acid sequence Cys-Hyp-Tyr(3×)-Xaa-Leu-Cys (SEQ ID NO:3), where 3× represents a halogen, nitro-, or trifluoromethyl group at the 3 position of the benzyl ring of the Tyrosine, where Hyp represents Hydroxyproline, and where Xaa can Gly or Asp. The fibrin binding moiety also can include the amino acid sequence Phe-His-Cys-Hyp-Tyr(3-I)-Asp-Leu-Cys-His-Ile-Leu (SEQ ID NO:4), where Tyr(3-I) represents 3-iodo-tyrosine and Hyp represents Hydroxyproline.

In some embodiments, the optical dye is covalently bound to the N-terminal amino acid of a peptide fibrin-binding moiety. The N-terminal amino acid can be a naturally-occurring or a non-naturally-occurring amino acid. For example, the N-terminal amino acid can be β-alanine (β-ala), 6-aminohexanoic acid (Ahx), or a lysine residue. The C-terminus of the fibrin binding moiety's amino acid sequence may be capped as a C-terminal amide. Alternatively, the C-terminus may be capped with a non-optical moiety. The C-terminal amino acid also can be in the D-configuration.

In other embodiments, the optical dye is covalently bound to the C-terminal amino acid of a peptide fibrin binding moiety. The N-terminus of the fibrin binding moiety's amino acid sequence may be alkylated. The N-terminal amino acid also can be in the D-configuration.

The optical dye can be selected from the group consisting of fluorescein, rhodamine, tetramethylrhodamine, hematoporphyrin, fluoresdamine, indocyanine, tetramethylrhodamine, Cosin, erythrosine, coumarin, methyl-coumarins, pyrene, Malacite green, stilbene, Lucifer Yellow, Cascade Blue, Texas Red, and derivatives thereof. In one embodiment, the optical dye is fluorescein. In another embodiment, the optical dye is tetramethylrhodamine.

Specific embodiments of optical agents for use in the method of the present invention include:

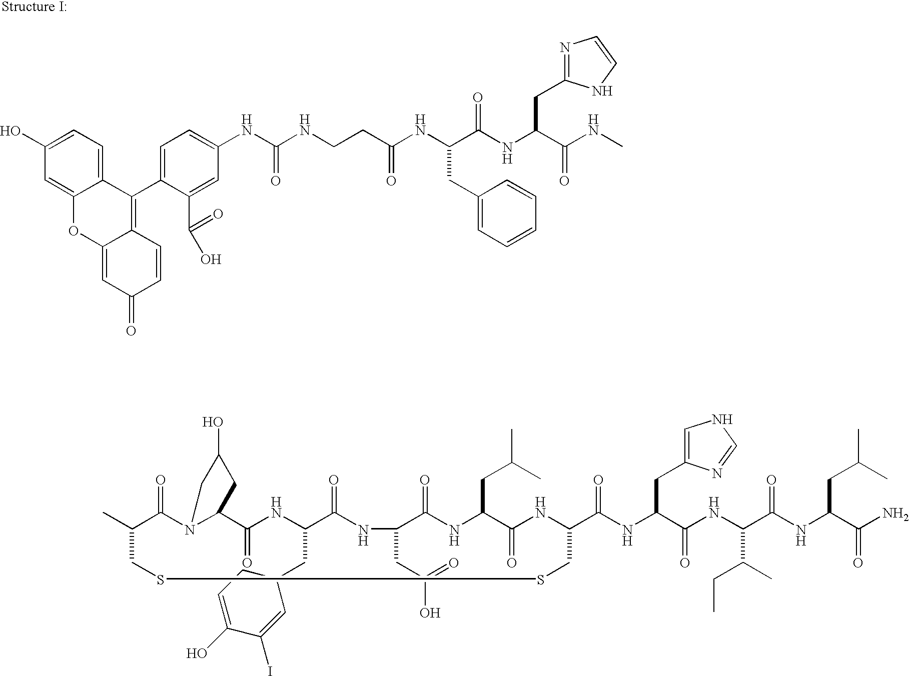

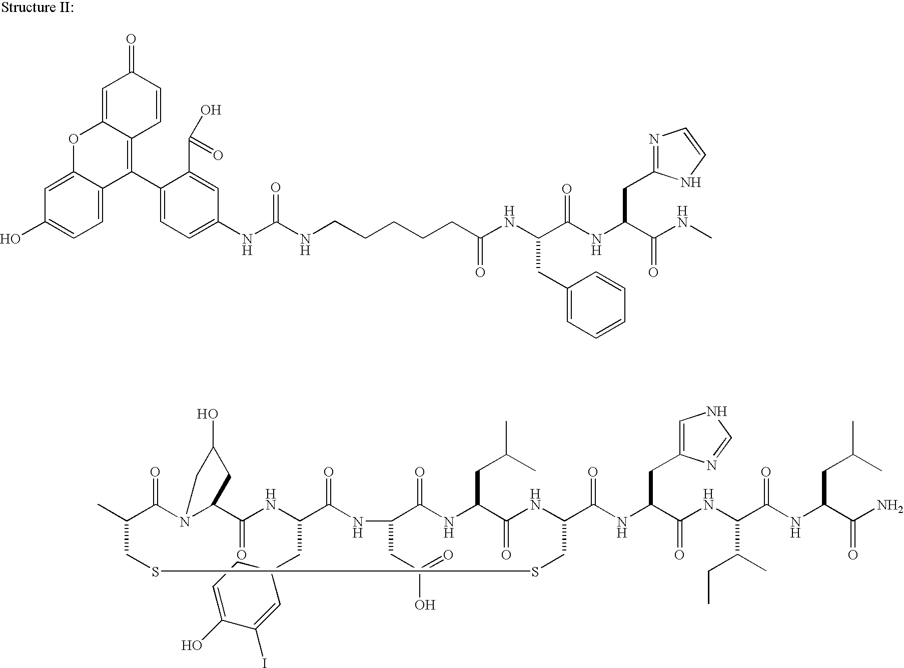

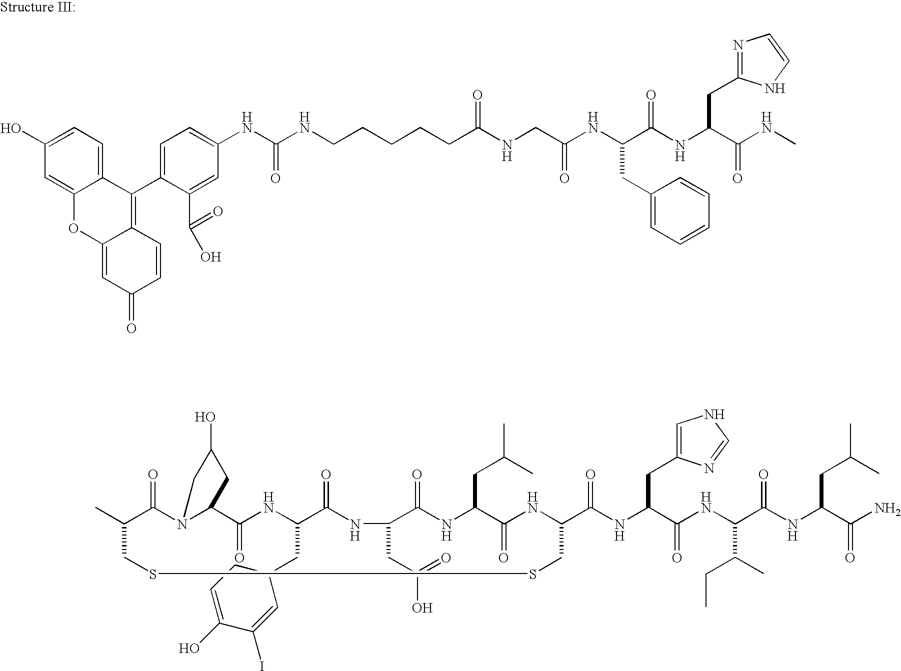

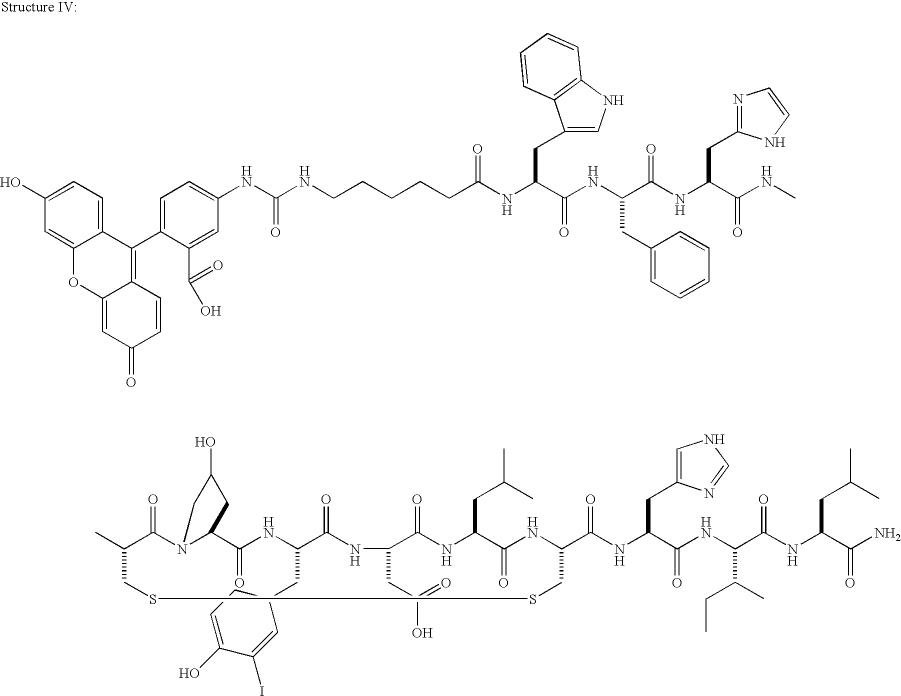

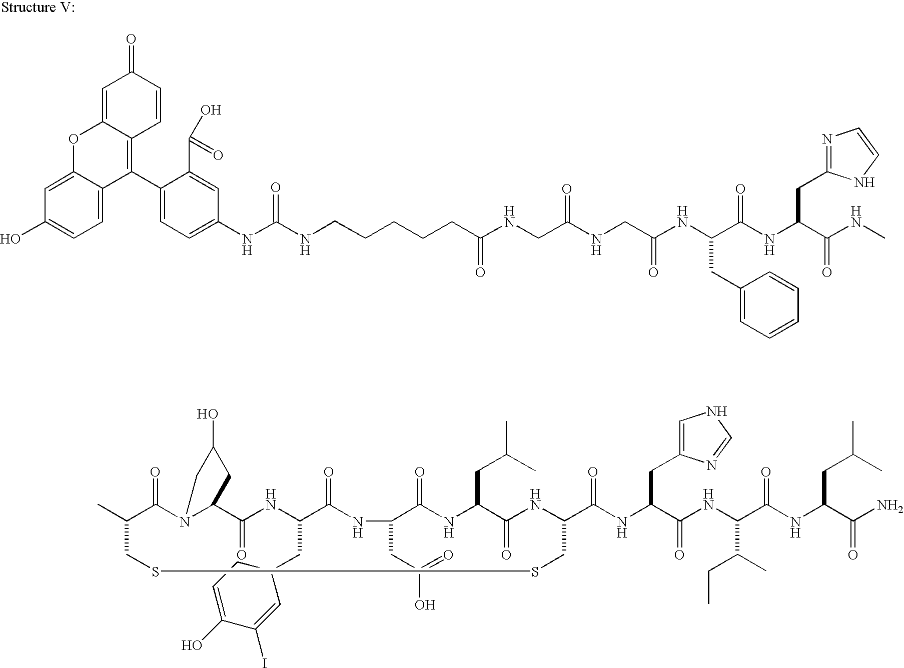

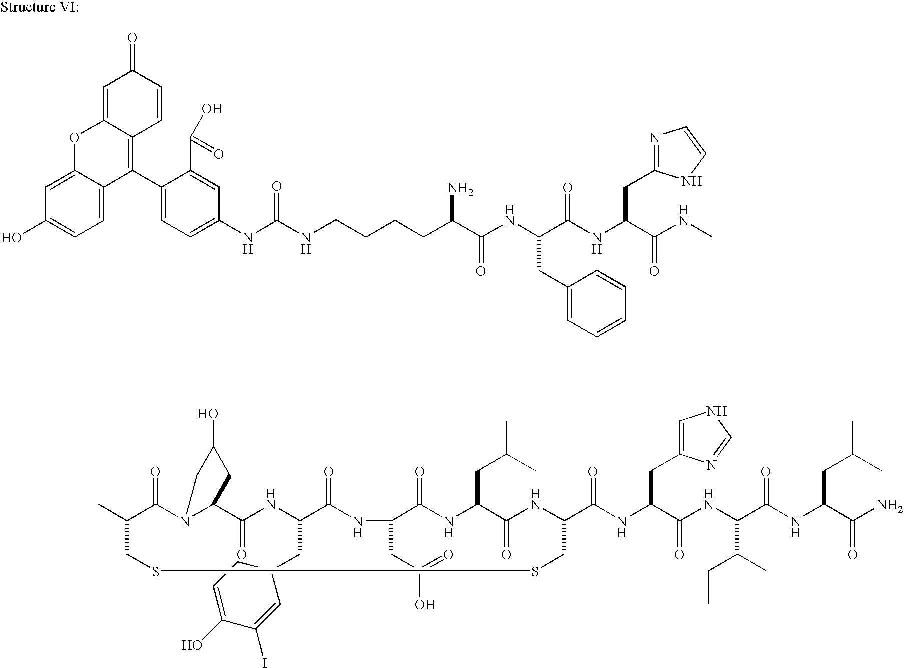

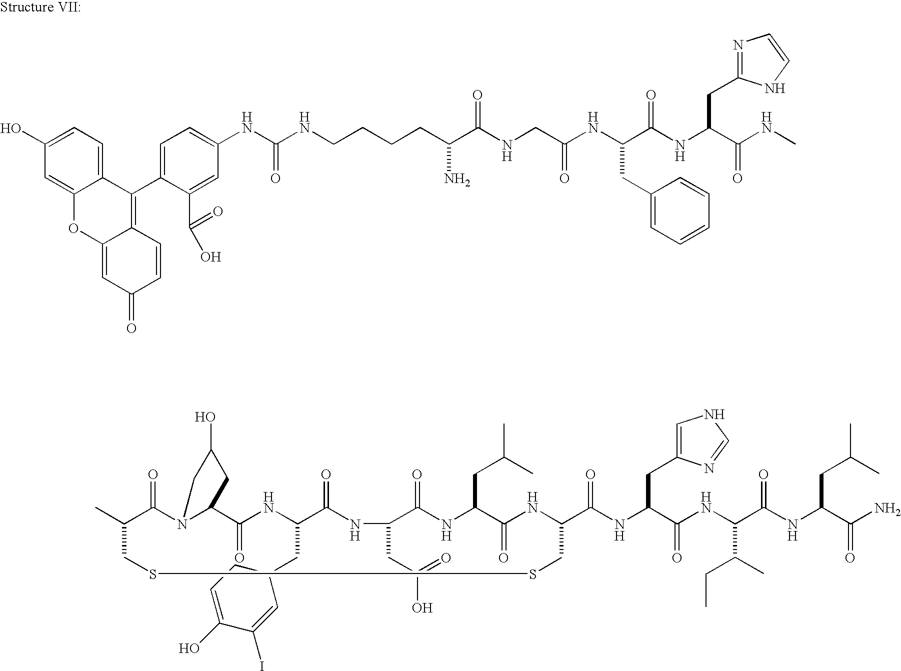

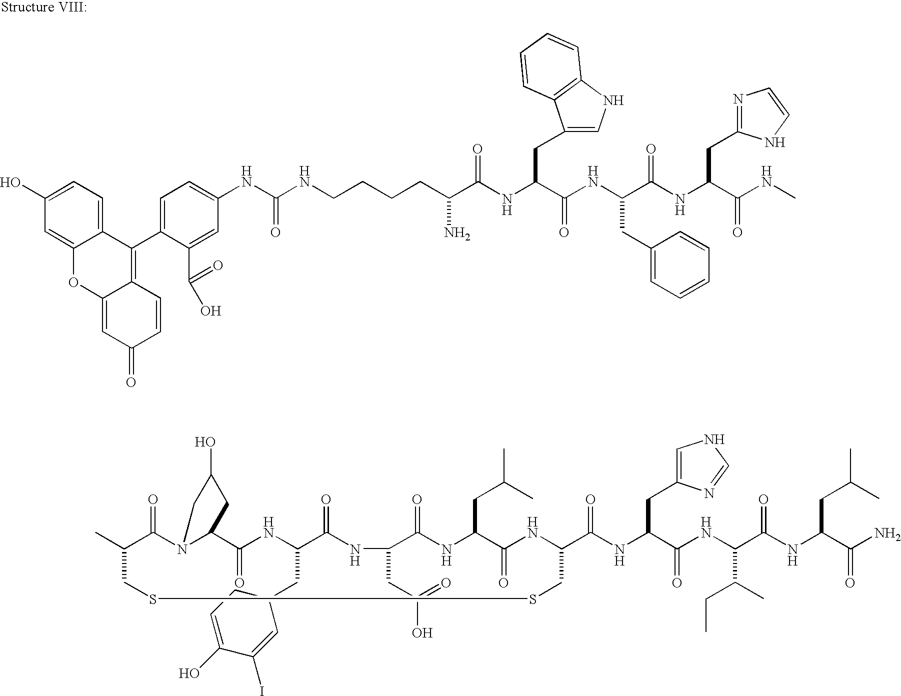

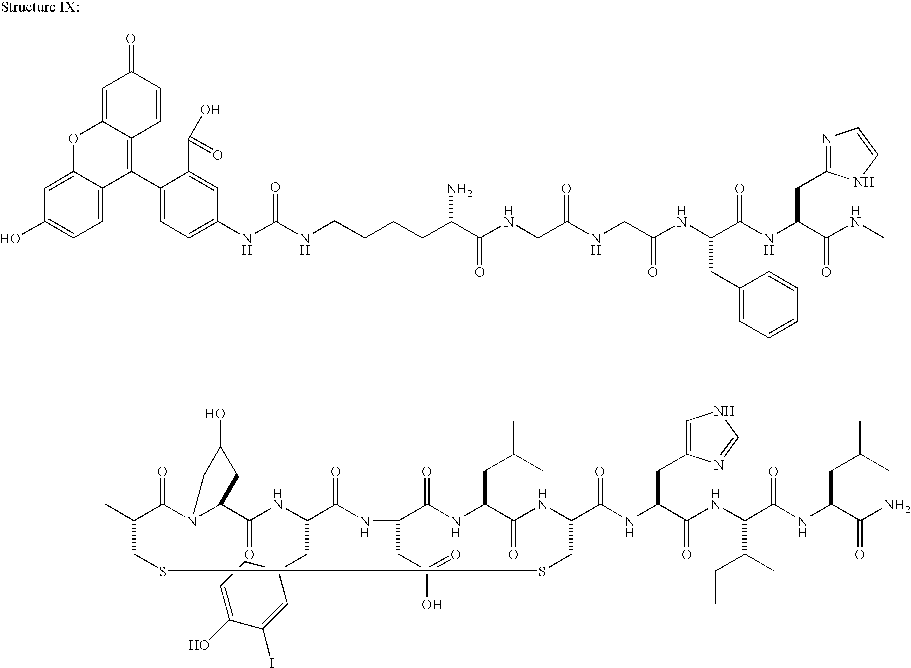

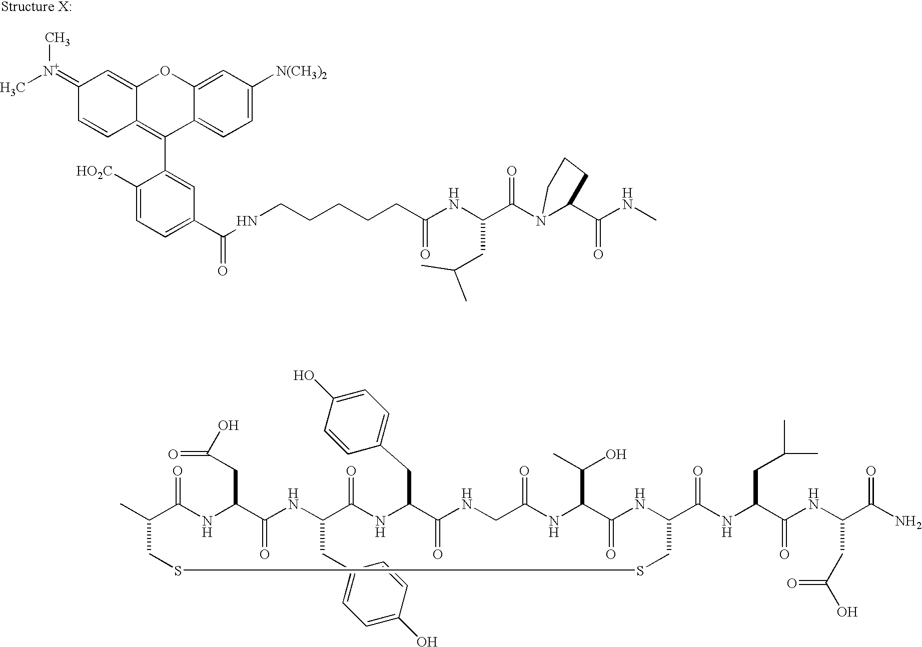

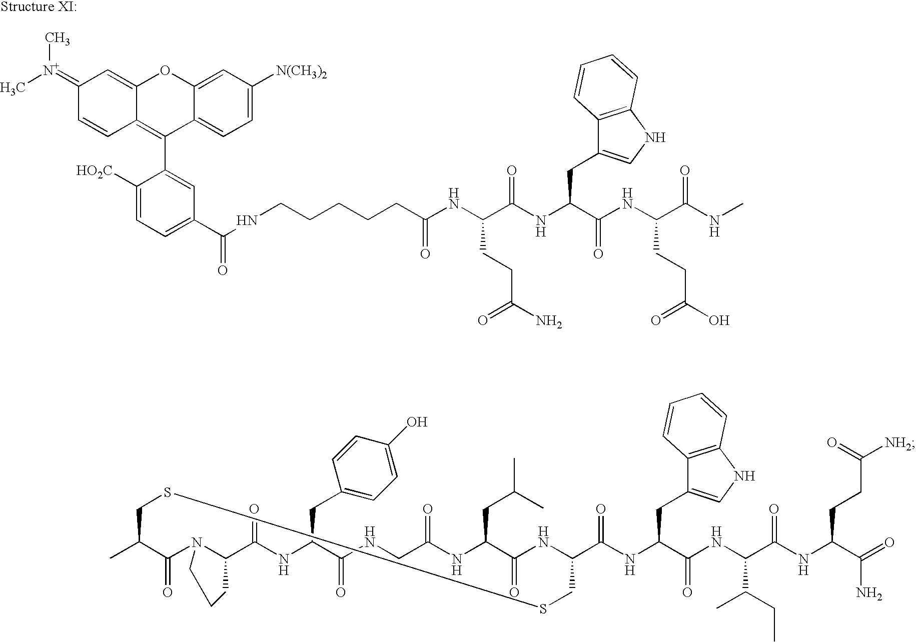

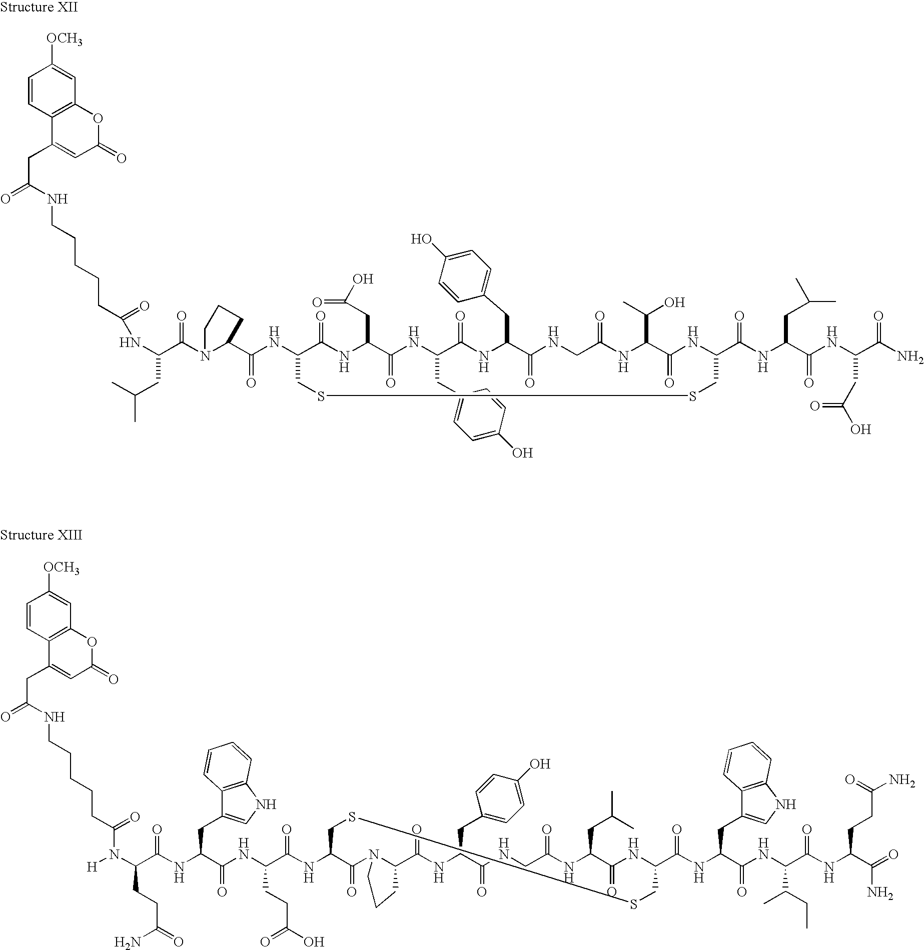

In one embodiment, the dissociation constant of the optical agent has a value less than about 10 μM. In another embodiment, the dissociation constant value of the optical agent is less than about 5 μM. Alternatively, the dissociation constant value of the optical agent is less than about 1 μM. The dissociation constant of the optical may also be less than about 0.3 μM.

The device inserted near the lesion may include a catheter and an optical detector, such as a fluorescence emission detector. The device may further include an excitation source. The device can be inserted near the lesion, in a cavity, a tissue, an interstitial space, or a blood vessel. In one embodiment, the device is inserted in the same blood vessel as the lesion.

The therapy can include a thrombolytic composition, such as tissue plasminogen activator (tPA), streptokinase, antistreplase, or urokinase. Alternatively, the therapy can include a mechanical manipulation of the lesion, such as by balloon angioplasty. In another embodiment, the therapy can include laser ablation of the lesion.

The therapy can be delivered by the device inserted near the lesion. Alternatively, in an embodiment where the therapy is a thrombolytic, the therapy can be administered intravenously at a site remote from the lesion. The therapy is delivered to at least a portion of the lesion. In one embodiment, the thrombolytic agent is delivered to about 90% of the surface of the lesion. In another embodiment, the thrombolytic is delivered to about 50% of the surface of the lesion. In yet another embodiment, the thrombolytic is delivered to about 10% of the surface of the lesion.

The method can include detecting the signal of the fibrin-optical agent complex during the delivery of the therapy. The method can include stopping the therapy delivery when the signal of the fibrin-optical agent complex decreases to a predetermined value. For example, in one embodiment, the therapy is stopped when the signal of the fibrin-optical agent complex is less than about 90% of the signal before delivery of the therapy. In another embodiment, the therapy is stopped when the signal of the fibrin-optical agent complex is less than about 50% of the signal before delivery of the therapy. In yet another embodiment, the therapy is stopped when the signal of the fibrin-optical agent complex is less than about 10% of the signal before delivery of the therapy.

In another aspect, the invention features compositions and kits that include an optical agent, wherein the optical agent includes an optical dye covalently linked to the N-terminus of a peptide fibrin binding moiety (FBM) via a linker, wherein the optical agent has the general formula: 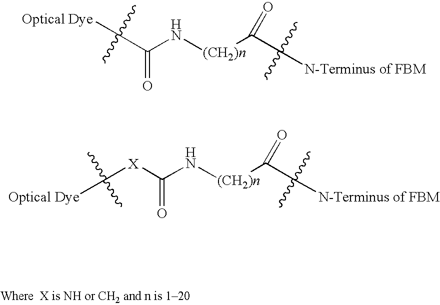
Particular embodiments of optical agents include structures I-XIII and pharmaceutically acceptable salts thereof.

The invention also features formulations that include compositions containing optical agents, wherein the formulation includes at least one ingredient selected from the group consisting of solubilizing agents, excipients, carriers, adjuvants, vehicles, preservatives, a local anesthetic, flavorings, and colorings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the methods, materials, and examples are illustrative only and not intended to be limiting.

Commonly used chemical abbreviations that are not explicitly defined in this disclosure may be found in The American Chemical Society Style Guide, Second Edition; American Chemical Society, Washington, D.C. (1997); “2001 Guidelines for Authors,” J. Org. Chem. 66(1), 24A (2001); and “A Short Guide to Abbreviations and Their Use in Peptide Science,” J. Peptide Sci. 5, 465-471 (1999).

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a table demonstrating the structures of embodiments of optical agents with their dissociation constants (Kd) to a DD(E) fragment of fibrin at 24° C.

FIG. 2 demonstrates a general synthetic scheme to couple an optical dye to the fibrin binding moieties of the present invention.

FIG. 3 provides the structures of two optical agents used in the imaging studies of Example 2.

### DETAILED DESCRIPTION

The invention provides optical agents and methods for detecting and treating intravascular lesions using the optical agents. Optical agents of the invention include an optical dye (OD) linked to a fibrin binding moiety (FBM), and have affinity for fibrin. After administration of an optical agent to a mammal (e.g., a human patient), the optical agent can form a fibrin-optical agent complex, which has a detectable signal, allowing for improved sensitivity of lesion detection. The affinity for fibrin is useful because fibrin is present in most lesions and can be targeted without interfering with normal thrombolytic processes.

The improved sensitivity allows for the detection of relatively small lesions and provides information about the presence and distribution of fibrin in the lesion. The use of optical agents and medical devices (e.g., a catheter) inserted near the lesion to detect the signal of fibrin-optical agent complexes also avoids the interference due to the background of flowing blood in a blood vessel. The medical devices inserted near the lesion may be used to deliver a therapy to at least a portion of the lesion in a timely and effective manner in order to reduce the size of the lesion or to alter the shape of the lesion. By monitoring the signal of the fibrin-optical agent complexes during the course of the therapy, the therapy may be stopped at a clinically significant timepoint.

Optical Agents

Optical agents of the invention include an OD and a FBM covalently bound to each other, either directly or via a linker (OD-L-FBM). The FBM can be a small molecule or a peptide. As used herein, the term “peptide” refers to a chain of amino acids that is about 2 to about 75 amino acids in length (e.g., 3 to 50 amino acids). Affinity of a peptide for fibrin can be expressed in terms of its dissociation constant (Kd), which is the equilibrium constant for the dissociation reaction of the peptide from the DD(E) fragment of fibrin. The term “DD(E) fragment of fibrin” refers to a fibrin subcomponent generated by proteolytic degradation of fibrin with plasmin or trypsin. The DD(E) fragment is a complex of the crosslinked D domains of adjacent fibrin monomers with the central E domain of fibrin (See, for example, Spraggon et al., Nature 389:455462 (1997)). Since DD(E) is a product resulting from the proteolysis of fibrin, one of skill in the art will understand that there may be some slight heterogeneity in its composition. The DD(E) fragment can be biotinylated and immobilized via avidin to a solid substrate (e.g., a multi-well plate). Peptides can be incubated with the immobilized DD(E) fragment in a suitable buffer and binding detected using known methodology. Methods for determining the dissociation constant of the peptide for DD(E) are set forth in WO 01/09188.

As a result of the FBM having affinity for fibrin, the optical agent also has affinity for fibrin. The term “affinity” refers to the capacity of the optical agent to be taken up by, retained by, or bound to the fibrin in the lesion. The affinity of an optical agent can be expressed in terms of its Kd, which is the equilibrium constant for the dissociation reaction of the optical agent from fibrin, and determined as discussed above for the peptide. The dissociation constant of the optical agent for DD(E) can have a value less than about 10 μM (e.g., 0.1 μM to 10 μM). In one embodiment, the dissociation constant value is less than about 5 μM. In another embodiment, the dissociation constant value is less than about 1 μM. The dissociation constant also may have a value less than about 0.3 μM (e.g., 0.2 μM).

Peptide fibrin binding moieties can include naturally occurring or non-naturally occurring amino acids. As used herein, the term “natural” or “naturally occurring” amino acid refers to one of the twenty most common occurring amino acids. Natural amino acids are referred to by their standard one- or three-letter abbreviations. The term “non-natural amino acid” or “non-natural” refers to any derivative of a natural amino acid including D forms, β and γ amino acid derivatives, α-N-alkylated amino acids, and amino acids having amine-containing side chains (such as Lys or Orn) in which the amine has been acylated or alkylated. It is noted that certain amino acids, e.g., hydroxyproline, that are classified as a non-natural amino acid herein, may be found in nature within a certain organism or a particular protein.

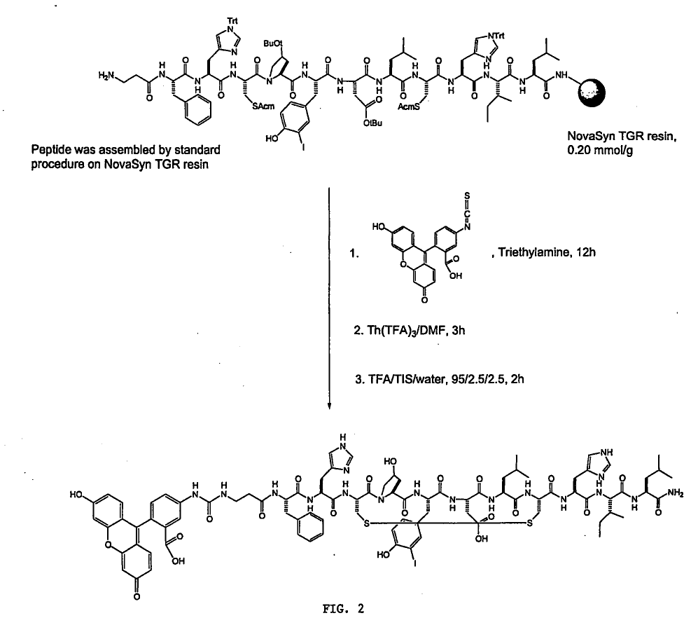

The fibrin binding moieties of the present invention may be cyclized or uncyclized. When cyclized, the fibrin binding moieties have a disulfide linkage between two cysteine residues in their amino acid sequence. Cyclization can occur using known methods, either before, during, or after modification of the FBM with the optical dye. See FIG. 2.

In some embodiments, the C or N-terminus of a fibrin binding moiety's amino acid sequence can be capped. For example, the C-terminus can be capped with an amide or the N-terminus can be capped by alkylating the amine group. Alternatively, the C or N-terminus can be capped using any non-optical moiety. The term “non-optical moiety” as used herein refers to any molecule that is not an optical dye. When the C or N-terminus is so capped, the optical agents of the present invention include one optical dye molecule per optical agent molecule. While not being bound by any theory, when the optical agent has only one optical dye molecule per optical agent molecule, the possibility of intramolecular quenching of the optical signal from one optical dye molecule to another optical dye molecule on the same optical agent molecule is eliminated.

The C and N-termini also can be rendered less susceptible to degradation, e.g., degradation by metabolic and proteolytic processes. For example, the C or N-terminal amino acid of the FBM may be a D-amino acid (i.e., having the “D” stereochemistry) in order to stabilize the FBM against degradation by proteases.

A FBM of the invention can include the amino acid sequence Cys-Asp-Tyr-Tyr-Gly-Thr-Cys (SEQ ID NO:1) or Cys-Pro-Tyr-Xaa-Leu-Cys (SEQ ID NO:2), wherein Xaa can be Gly or Asp. In another embodiment, the fibrin binding moiety includes the amino acid sequence Cys-Hyp-Tyr(3×)-Xaa-Leu-Cys (SEQ ID NO:3), where 3× represents a halogen, nitro-, or trifluoromethyl group at the 3 position of the benzyl ring of the Tyrosine, Hyp represents Hydroxyproline (e.g., 4-hydroxyproline), and Xaa is Gly or Asp. In yet another embodiment, the fibrin binding moiety includes the amino acid sequence Phe-His-Cys-Hyp-Tyr(3-I)-Asp-Leu-Cys-His-Ile-Leu (SEQ ID NO:4), where Tyr(3-I) represents 3-iodo-tyrosine and Hyp represents Hydroxyproline.

Peptide fibrin binding moieties can be synthesized using known peptide synthesis methods, including solid phase synthesis. Amino acids with many different protecting groups appropriate for immediate use in solid phase synthesis of peptides are commercially available. Example 1 demonstrates the synthesis of a FBM using a solid phase synthesis method. Additional methods and details for the synthesis of the fibrin binding moieties may be found in WO 01/09188.

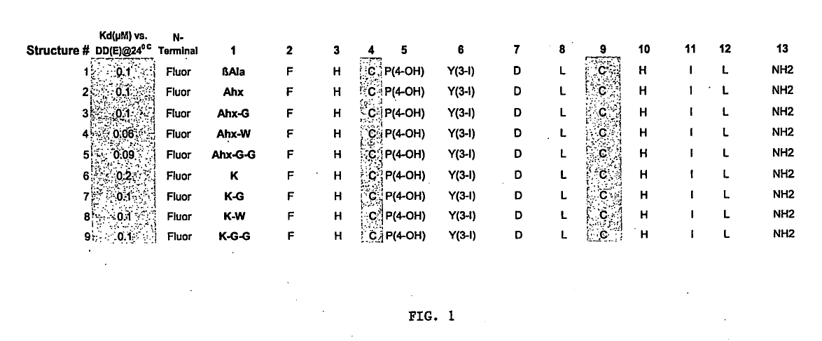

Once synthesized, a FBM can be covalently coupled to an optical dye. For example, the optical dye can be covalently bound to the N-terminal amino acid of a FBM (e.g., a β-alanine, 6-aminohexanoic acid, or lysine residue, see FIG. 1), the C-terminal amino acid of a FBM (e.g., a leucine residue), or to both the N and C-termini of a FBM. In some embodiments, it is preferred that when the C-terminus is linked to an OD, the N-terminus is not covalently bound to or capped with an OD. The OD provides an optical signal that allows the FBM to be detected (e.g., by a fluorescence emission spectrum). Any OD may be used, provided it does not render the optical agent pharmaceutically unacceptable. Non-limiting examples of suitable OD include fluorescein, rhodamine, hematoporphyrin, fluoresdamine, indocyanine, tetramethylrhodamine, Cosin, erythrosine, coumarin, methyl-coumarins, pyrene, Malacite green, stilbene, Lucifer Yellow, Cascade Blue®, Texas Red® (Molecular Probes, Inc., Eugene, Oreg.), and derivatives thereof. Fluorescein and tetramethylrhodamine are particularly useful ODs. FIG. 2 demonstrates a method for the modification of a FBM with the optical dye fluorescein.

In some embodiments, the OD is covalently linked to a FBM via a linker. Suitable linkers can be peptidic or non-peptidic in nature, and can be an all-carbon chain, or can contain heteroatoms such as, e.g., oxygen, nitrogen, sulfur, and phosphorus. The linker can be a linear or branched chain, or can include structural elements such as phenyl ring(s), non-aromatic carbocyclic or heterocyclic ring(s), double or triple bond(s), and the like. Linkers may be substituted with alkyl, aryl, alkenyl, or alkynyl groups.

In some embodiments, a linker can have one of the following formulas:

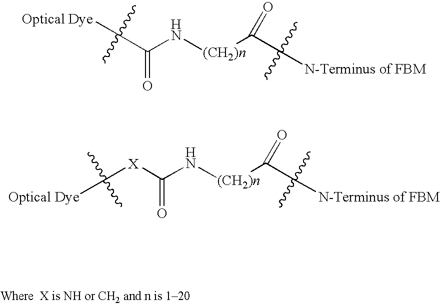

Optical agents of the invention may contain one or more asymmetric carbon atoms and thus may occur as racemates and racemic mixtures, single enantiomers, diastereomeric mixtures, and individual diastereomers. All such isomeric forms of these compounds are included in the present invention. Although the specific compounds exemplified in this application may be depicted in a particular stereochemical configuration, compounds having either the opposite stereochemistry at any given chiral center or mixtures thereof are also envisioned.

Specific embodiments of optical agents for use in the method of the present invention are shown in FIG. 1. In FIG. 1, “fluor” indicates a fluorescein as the OD; Ahx indicates 6-aminohexanoic acid; β-ala indicates β-alanine; P(4-OH) indicates Hydroxyproline (Hyp); and Y(3-I) indicates 3-Iodo-Tyrosine. Single capital letters in the table correspond to the single amino acid letter code. The NH2 in position 13 indicates that the C-terminus of amino acid number 12 is capped as an amide. FIG. 1 provides the Kd (μM) vs. DD(E) for each optical agent.

Specific structures corresponding to FIG. 1 include:

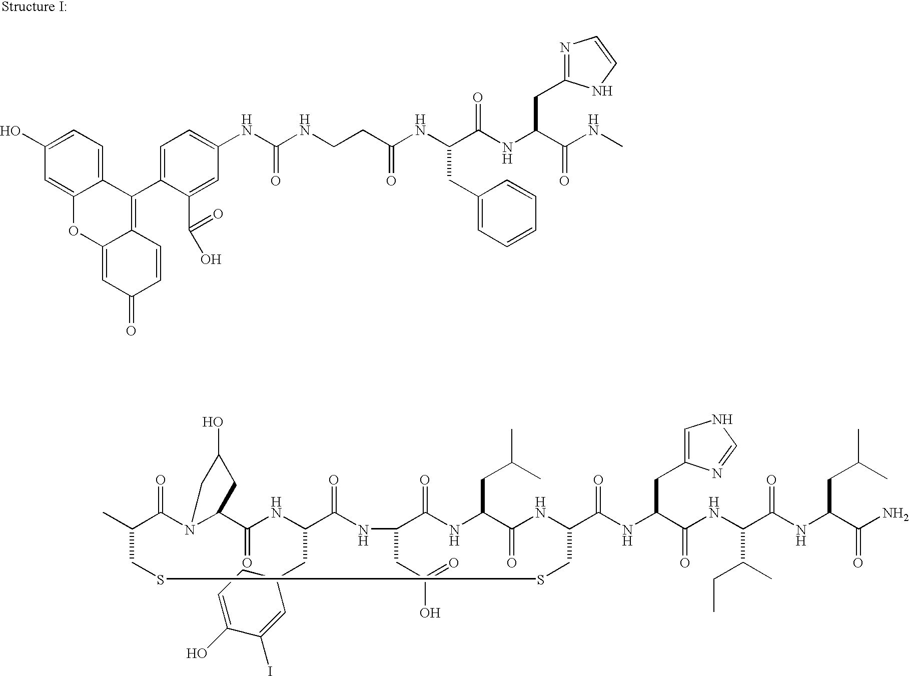

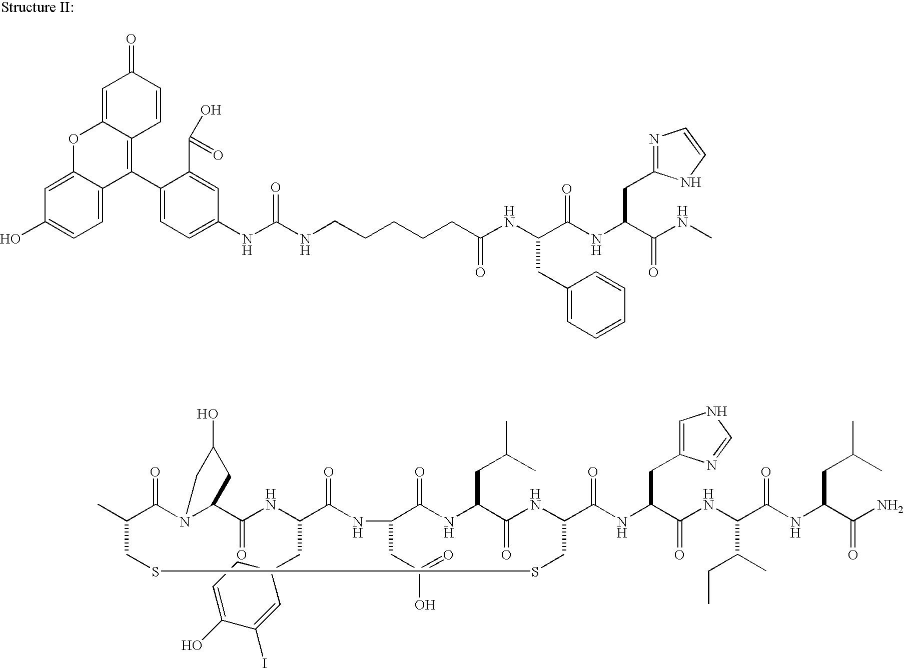

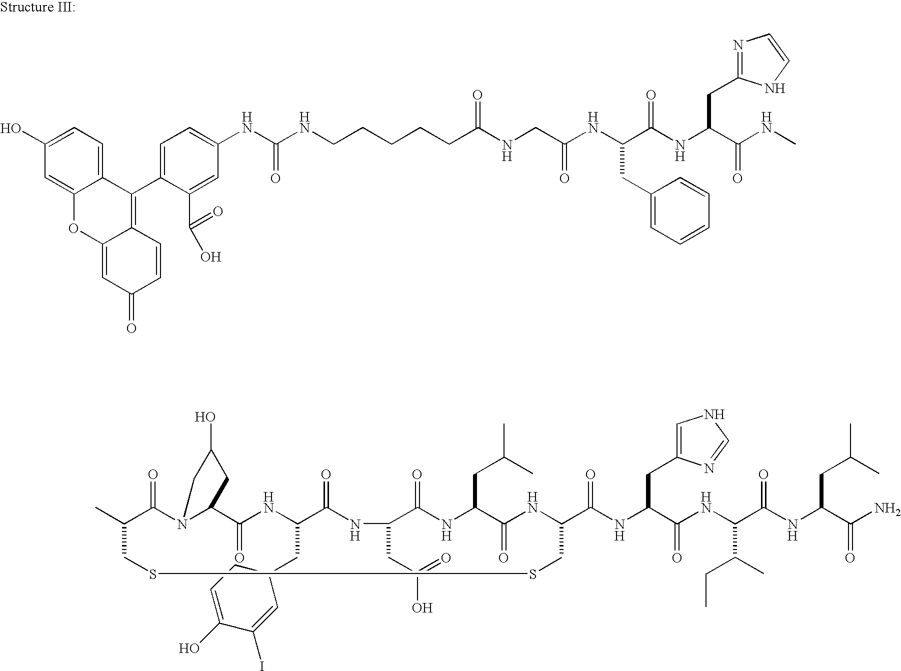

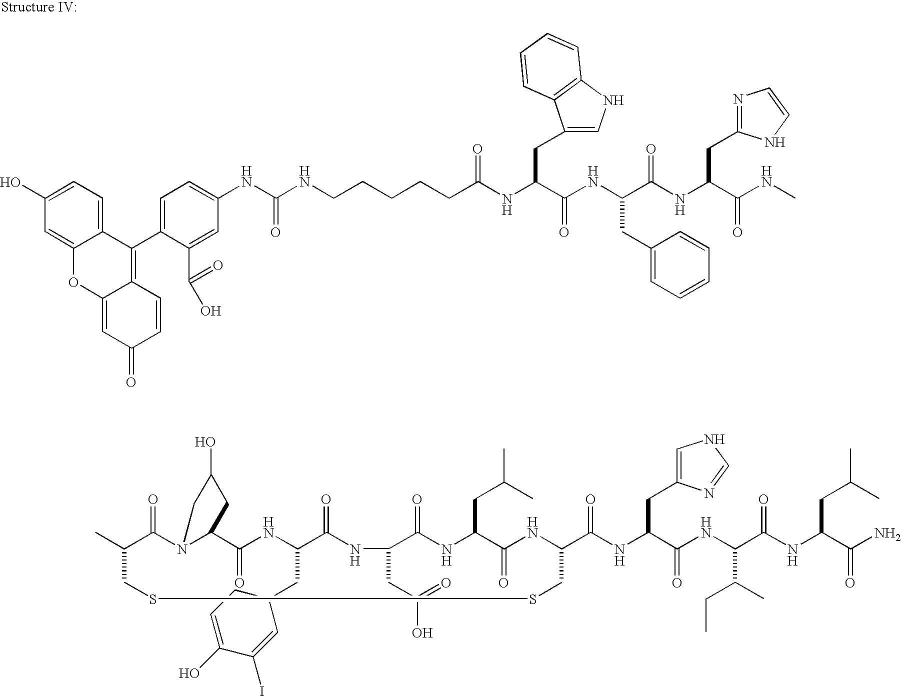

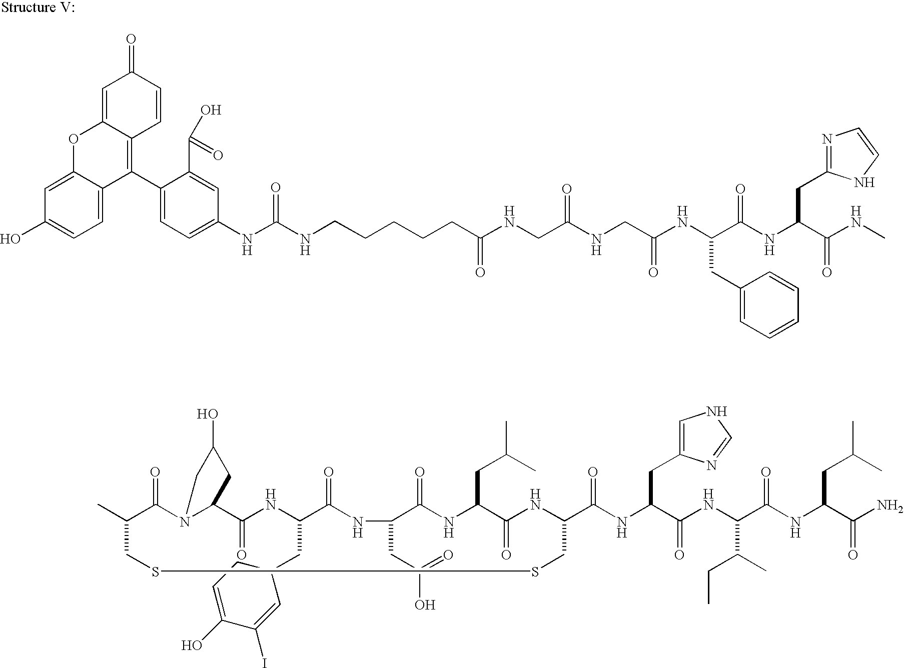

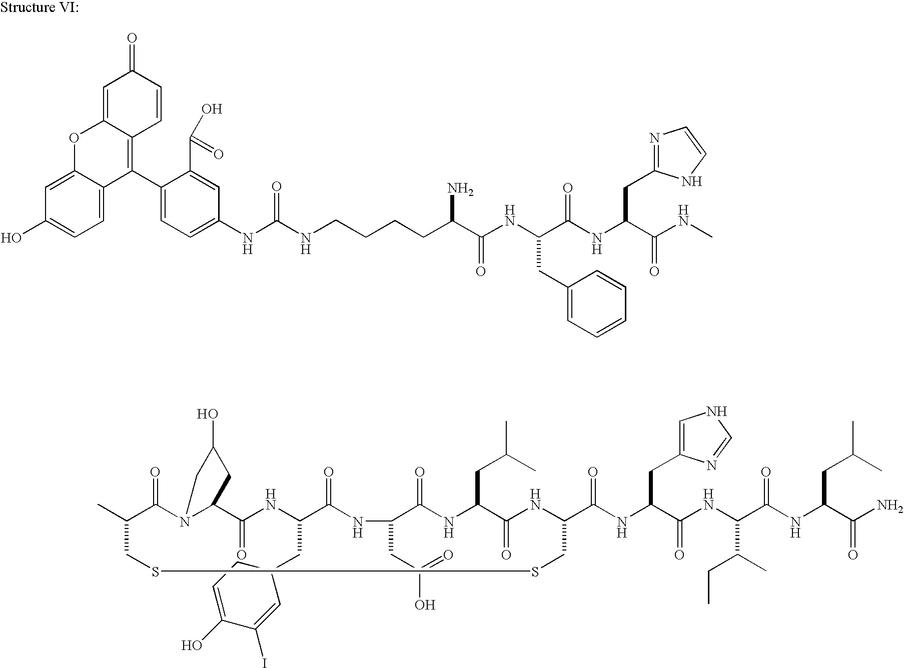

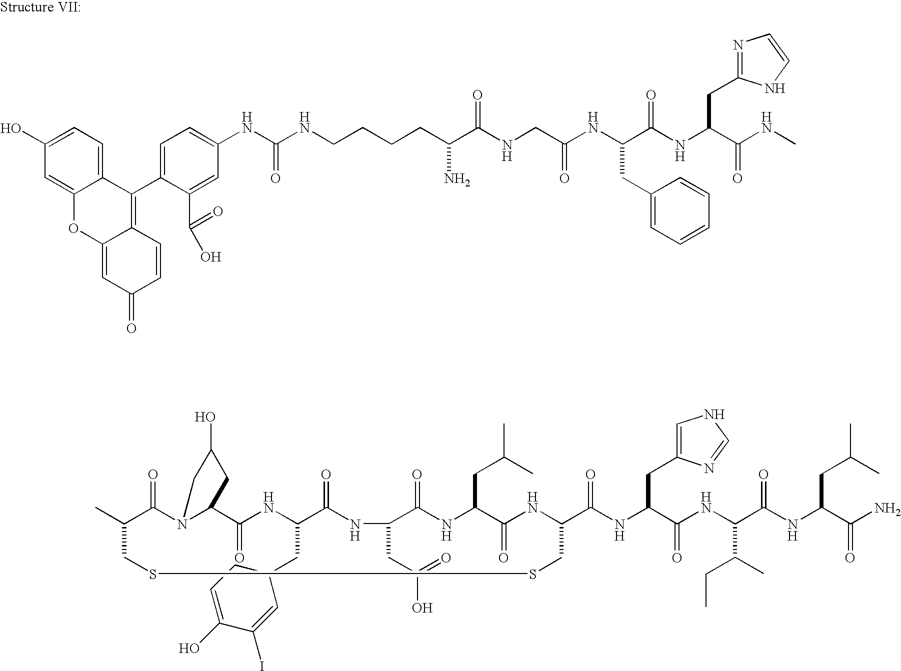

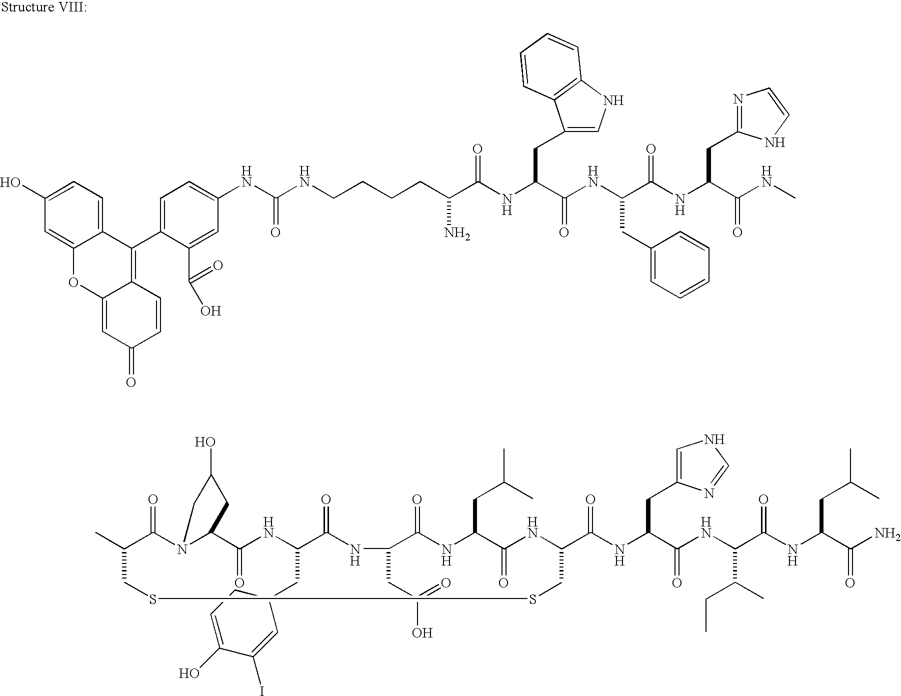

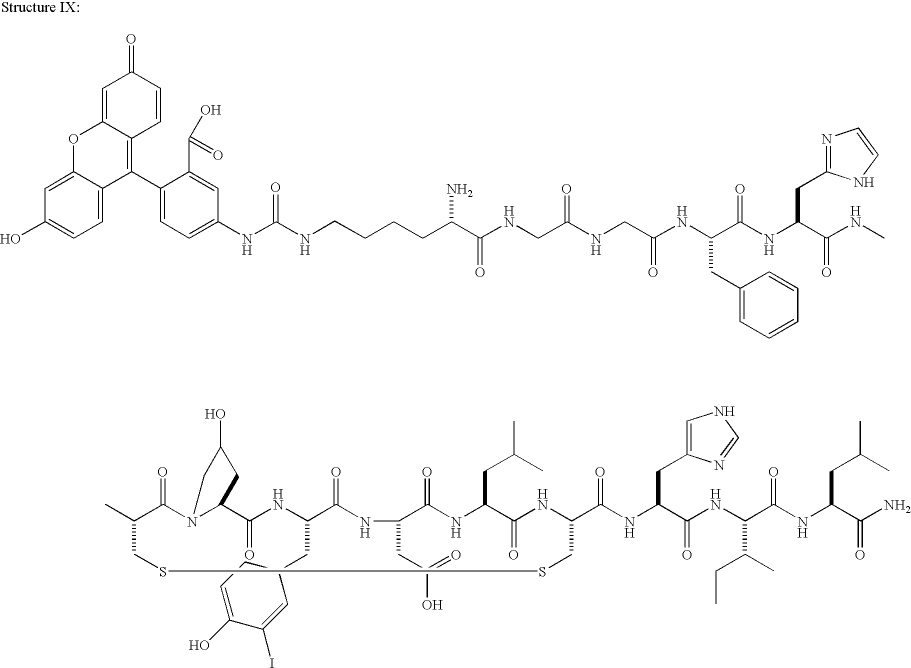

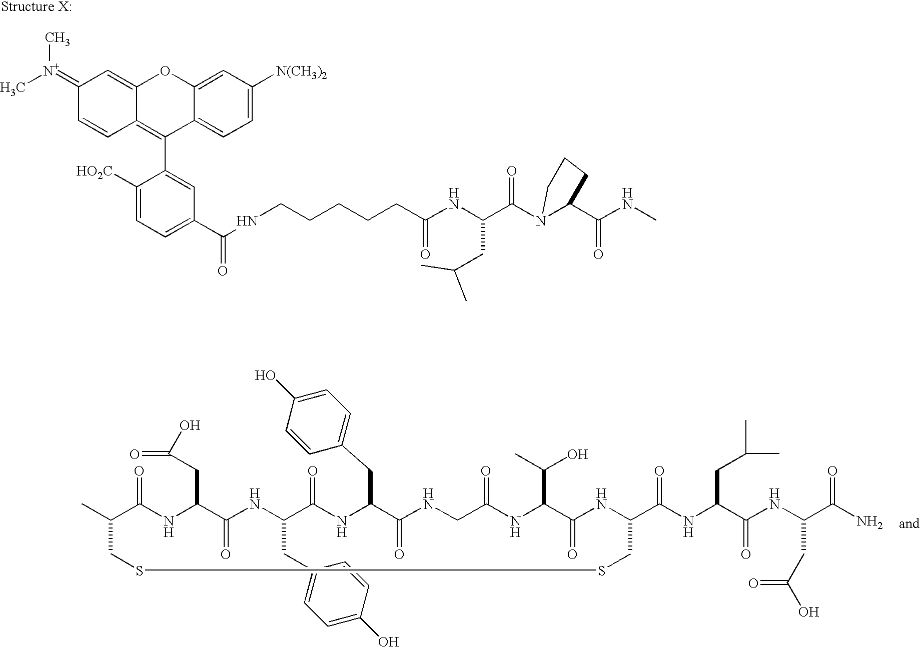

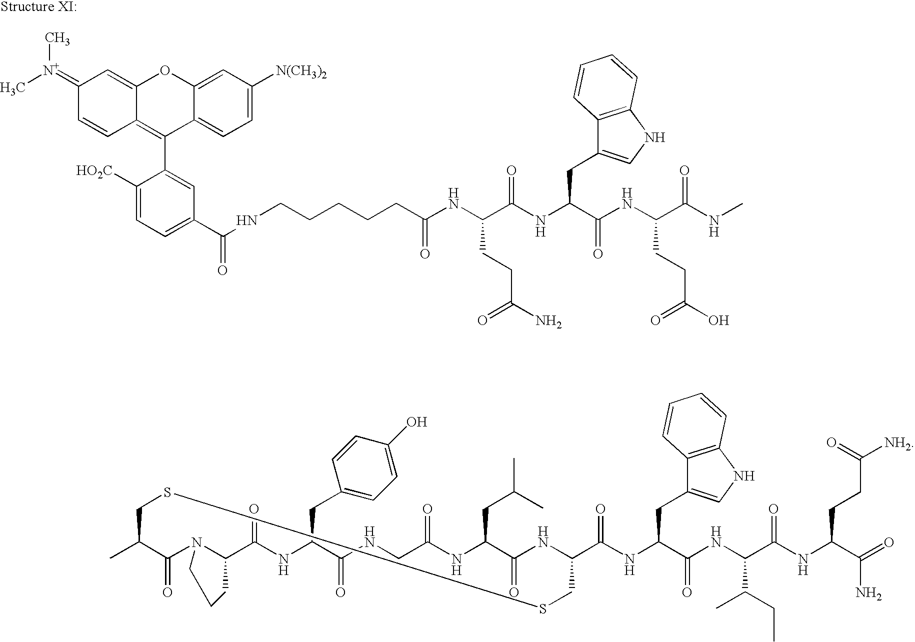

Additional examples of optical agents include Structures XII and XIII (shown below). In structures XII and XIII, the OD is a coumarin dye. The Kd vs. DD(E) of structure XII is 6.6 μM; the Kd vs. DD(E) of structure XIII is 0.2 μM. 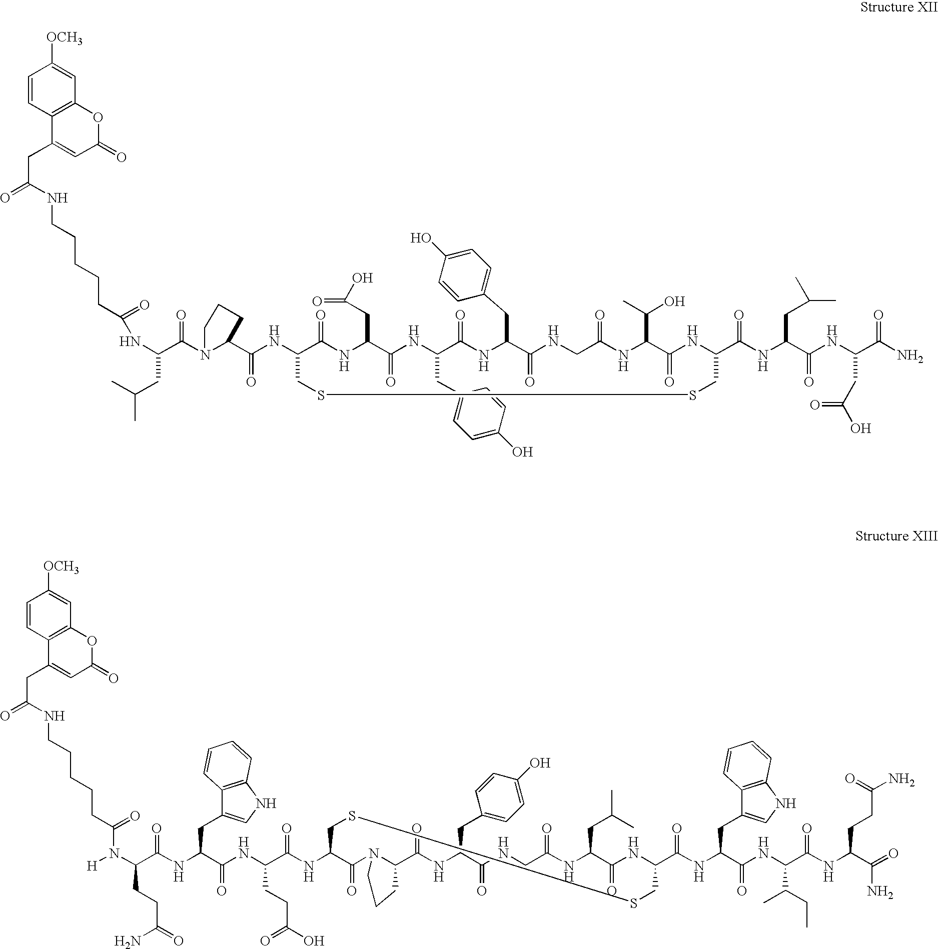
Methods of Treating Intravascular Lesions

According to one aspect of the invention, a method is provided to treat an intravascular lesion. The term “intravascular lesion” means a lesion within a blood vessel. “Blood vessel” as used herein can include arteries, veins, capillaries, and chambers of the heart. The lesion can be a thrombus, a clot, an atherosclerotic plaque, or an embolus. In particular, the lesion can be a deep vein thrombus, a coronary thrombus, a carotid thrombus, an atherosclerotic plaque, including plaque characterized as high risk, an atrial or ventricular thrombus, an aortic arch thrombus, or a pulmonary embolus.

The lesion may include fibrin on its surface. The exposed fibrin may be in contact with blood flowing in the blood vessel. While not being bound by any theory, it is believed that the optical agent can form a fibrin-optical agent complex more efficiently when there is exposed fibrin on the lesion's surface. In addition, while again not being bound by any theory, it is believed that lesions with exposed fibrin are at the highest risk for spontaneous dislodging.

The method includes administering an optical agent or a derivative thereof. Suitable optical agent derivatives include any pharmaceutically acceptable salt, ester, or other derivative of a composition of this invention, which, upon administration, is capable of providing (directly or indirectly) a compound of this invention or an active metabolite or residue thereof. Other derivatives are those that increase the bioavailability of the compounds when administered or which enhance delivery to a particular biological compartment.

Optical agents or derivatives thereof are formulated in a pharmaceutically acceptable manner such that the agent can be administered to a patient or animal without unacceptable adverse effects The optical agent can be formulated in accordance with routine procedures as a pharmaceutical formulation adapted for human patients or animals. Where necessary, the formulation can include such ingredients as solubilizing agents, excipients, carriers, adjuvants, vehicles, preservatives, a local anesthetic, flavorings, colorings, and the like. The ingredients may be supplied separately, e.g., in a kit, or mixed together in a unit dosage form. The dosage to be administered and the mode of administration will depend on a variety of factors including age, weight, sex, condition of the patient, pharmacokinetic parameters of the formulation, genetic factors, and the like. As one of skill in the art will recognize, the dosage will ultimately decided by the clinician.

The optical agent can be administered in any number of conventional ways, including orally or parenterally (e.g., subcutaneously, intravenously, intraarterially, interstitially, intrathecally, or intracavity administration). After administration, the optical agent forms a fibrin-optical agent complex at the site of the lesion. The affinity of the optical agent for fibrin allows the agent to localize at the fibrin within or on the lesion. The fibrin-optical agent complexes have an optical signal that can be detected. For example, the optical signal may be a fluorescence emission spectrum. The optical signal from the fibrin-optical agent complexes may be the same as or different from the optical signal of the optical agent before administration. For example, the signal may undergo a shift, a reduction, or an enhancement in a fluorescence wavelength maximum. The signal can be any optical signal that can be detected, including transmission or absorption of a particular wavelength of light, fluorescence or phosphorescence absorption and emission, reflection, changes in absorption amplitude or maxima, and elastically scattered radiation.

A device is inserted near the lesion to obtain information (i.e., data) about the lesion based on detecting a signal of the fibrin-optical agent complexes. A catheter such as the OPTICATH® family of fiber-optic catheters sold by Abbott Laboratories can be used for detecting the signal from the fibrin-optical agent complex. These catheters also can optionally be used for delivering the therapy to the lesion. Other possible fiber-optic catheters can be obtained from COOK and Baxter Healthcare corporations. Fiber-optic catheters from the Wellman Laboratories of Photomedicine at the Massachusetts General Hospital are suitable for detecting the fibrin-optical agent complex. FISO Technologies has high quality fiber-optic sensors designed for insertion into catheters. Other possible fiber-optic catheter detection systems are disclosed in U.S. Pat. Nos. 4,175,545; 4,416,285; 4648,892; 5,015,463; and 6,366,726.

The general position of the lesion typically is determined before the device is inserted nearby. The general position of the lesion may be determined by detecting the fibrin-optical agent complexes with a detector outside of the body of the patient. For example, if the optical agent includes a fluorescent optical dye, the location of the fluorescence emission of the optical dye, which generally corresponds to the location of the fibrin-optical agent complexes, may be determined with a fluorescence detector located outside the body of the patient. Alternatively, the position for device insertion is determined by reference to any number of known methods to determine the general location of a lesion, including the use of magnetic resonance or radionuclide-labeled agents that target a lesion, X-ray angiographic techniques, ventilation-perfusion scans of the lungs, and the like.

In another embodiment, the general position of the lesion may be determined by knowledge of the location where a lesion has occurred in the past. For example, the general position of a lesion can be estimated by reference to the medical history of the patient, e.g., the location where a stent or angioplastic procedure had been performed in the past, or the location where the patient is known to have experienced lesions in the past.

The device is inserted near the lesion. The device may be inserted into a cavity, a tissue, an interstitial space, or a blood vessel. In one embodiment, the device is inserted in the same blood vessel as the lesion. For example, if the device is a catheter, the catheter may be placed within 10 cm of the lesion. Alternatively, the catheter may be placed within 5 cm of the lesion. Alternatively, the catheter may be placed within 1 cm of the lesion.

Information about the lesion is based on detecting a signal of the fibrin-optical agent complex. The device inserted near the lesion may include an optical detector to detect the signal of the fibrin-optical agent complex. In one embodiment, the device includes a fluorescence emission detector. The device can also include an excitation source. The excitation source can provide the excitation wavelength of light, if necessary, to result in the optical signal generated by the fibrin-optical agent complex and detected by the optical detector. For example, excitation of the optical dye fluorescein occurs at 492 nm, while emission is detected at 515 nm. Excitation of the optical dye tetramethylrhodamine occurs at 555 nm, while emission is detected at 575 nm.

The information about the lesion that is obtained based on the detection of the signal of fibrin-optical agent complexes can include the size and shape of the lesion; the surface features of the lesion; the distribution and relative amount of fibrin within the lesion, including the amount exposed on the surface of the lesion; an assessment of the risk profile (e.g., ability to dislodge spontaneously) of the lesion; and an estimate of vessel occlusion and stenosis.

After the information about the lesion is obtained, a therapy is delivered based on the obtained information to at least a portion of the lesion. The therapy can be delivered by the device inserted near the lesion. For example, if the device is a catheter, the catheter can deliver the therapy to the lesion nearby. Alternatively, in an embodiment where the therapy is a thrombolytic, the therapy can be delivered intravenously at a site remote from the lesion. In one embodiment the therapy is delivered to about 90% of the surface of the lesion. In another embodiment, the therapy is delivered to about 50% of the surface of the lesion. In yet another embodiment, the therapy is delivered to about 10% of the surface of the lesion.

The therapy should either reduce the size of the lesion or alter the shape of the lesion. To reduce the size of the lesion, the therapy can include a thrombolytic composition such as tissue plasminogen activator (tPA), streptokinase, antistreplase, or single or two-chain urokinase. Additional information concerning the use of thrombolytics, including dosage, formulation, and time course of treatment are set forth in WO 01/09811.

To reduce the size of the lesion or to alter its shape, the therapy can include a mechanical manipulation of the lesion. For example, the inserted device can include components to perform a balloon angioplasty at the site of the lesion. Balloon angioplastic treatment of a lesion can reduce the size of the lesion or alter its shape by flattening it against the blood vessel to allow blood flow. Angioplasty, sometimes called “PTCA” (percutaneous transluminal coronary angioplasty) represents the majority of interventional procedures. In this procedure, a catheter is inserted near the site of the lesion, and a tiny balloon is inflated. These devices compress the lesion against the artery wall, and open the artery, thus allowing increased flow. See, for example, Kandarpa K, et al., “Transcatheter interventions for the treatment of peripheral atherosclerotic lesions: part II,” Journal of Vascular & Interventional Radiology. 12(7):807-12 (2001 July); Kandarpa K, et al., “Transcatheter interventions for the treatment of peripheral atherosclerotic lesions: part I,” Journal of Vascular & Interventional Radiology. 12(6):683-95 (2001 June).

In another embodiment, the therapy can include laser ablation of the lesion. For example, the inserted device can include components to perform laser ablation at the site of the lesion. See for example, the web site of the American Heart Organization (Heart and Stroke A to Z Guide), wherein it is noted that: “laser angioplasty is a technique used to open coronary arteries blocked by plaque (the build-up of cholesterol and other fatty substances in the inner lining of an artery). A catheter (thin tube) with a laser at the tip is inserted into an artery and advanced through the blood vessels to the blocked artery in the heart. The laser emits pulsating beams of light that vaporize the plaque. This procedure has been used alone and with balloon angioplasty.”

The method can further include detecting the signal of the fibrin-optical agent complex during the delivery of the therapy. As the therapy is delivered, the inserted device continues to detect the signal of the fibrin-optical agent complex. The clinician can determine, based on the signal detected, when to stop delivery of the therapy. The method can include stopping the therapy delivery when the signal of the fibrin-optical agent complex decreases to a predetermined value. For example, in one embodiment, the therapy is stopped when the signal of the fibrin-optical agent complex is less than about 90% of the signal before delivery of the therapy. In another embodiment, the therapy is stopped when the signal of the fibrin-optical agent complex is less than about 50% of the signal before delivery of the therapy. In yet another embodiment, the therapy is stopped when the signal of the fibrin-optical agent complex is less than about 10% of the signal before delivery of the therapy.

In the methods of the present invention, the optical agent forms a fibrin-optical agent complex at the site of the lesion. The ability of the optical agent to form a fibrin-optical agent complex may be measured by examining the optical agent's dissociation constant (Kd) for a DD(E) fragment of fibrin as discussed above.

Articles of Manufacture

Optical agents described herein can be combined with packaging material and sold as articles of manufacture or kits. Components and methods for producing articles of manufactures are well known. The articles of manufacture may combine one or more optical agents described herein. In addition, the articles of manufacture may further include one or more of the following: sterile water or saline, pharmaceutical carriers, buffers, syringes, or catheters. A label or instructions describing how the optical agent can be used for treating an intravascular lesion may be included in such kits. The optical agents may be provided in a pre-packaged form in quantities sufficient for single or multiple administrations.

The invention is further described in the following examples, which do not limit the scope of the invention described in the claims.

### EXAMPLES

### Example 1

### Synthesis of Optical Agents

Preparation of Fibrin Binding Moiety-Solid Phase Synthesis

NovaSyn TGR resin (0.20 mmol/g, 100 mg, 20 μmol) was washed with NMP/ether/NMP. The peptide was assembled by the standard solid phase method using the PyBOP/HOBt/DIEA activation. After the coupling of the final amino acid residue, the resin bound peptide was treated with a solution of piperidine in DMF (20% by volume, 2.0 mL) for 10 minutes to remove the Fmoc protecting group. The resin was washed thoroughly with NMP/ether/NMP, and was treated with a solution of fluoroscein-5-isothiocyanate (23.4 mg, 60 μmol) and diisopropylethylamine (11.6 mg, 15.7 μL, 90 μmol) in DMF (1.5 mL) for 12 hours. The resin was washed thoroughly (NMP/ether/NMP), and treated with a solution of Tl(TFA)₃ (18.7 mg, 34.5 μmol) in DMF (1.5 mL) at 4° C. for three hours. The resin was washed after this treatment, and treated with a cocktail of TFA/TIS/water (95/2.5/2.5, 2.0 mL) for two hours. The crude peptide was precipitated by adding ether to the cleavage cocktail and purified by preparative HPLC using a Vydac C-18 column.

TMR (tetramethylrhodamine) derivatives were prepared using 6-carboxytetramethylrhodamine, succinimidyl ester instead of fluoroscein-5-isothiocyanate.

Modification of Fibrin Binding Moiety with Optical Dye. See FIG. 2.

Mass Spectrometry and Kd Data of Structures I-XI:

| | | MS data | Kd (μM) vs. |
|---|---|---|---|
| Compound | MS data[(M + 2H)/2]+ | (M + H)+ | DD(E) @ 24° C. |
| Structure I | 972.5 | N/a | .1 |
| Structure II | 993 | N/a | .1 |
| Structure III | 1022.3 | N/a | .1 |
| Structure IV | 1086.7 | N/a | .06 |
| Structure V | 1050.8 | N/a | .09 |
| Structure VI | 1001.2 | N/a | .2 |
| Structure VII | 1029.9 | N/a | .1 |
| Structure VIII | 1094.3 | N/a | .1 |
| Structure IX | 1058.6 | N/a | .1 |
| Structure X | N/a | 1795 | N/a |
| Structure XI | N/a | 2049 | 0.09 |

N/a = not available

### Example 2

### Detection of Fibrin-Optical Agent Complex on a Lesion

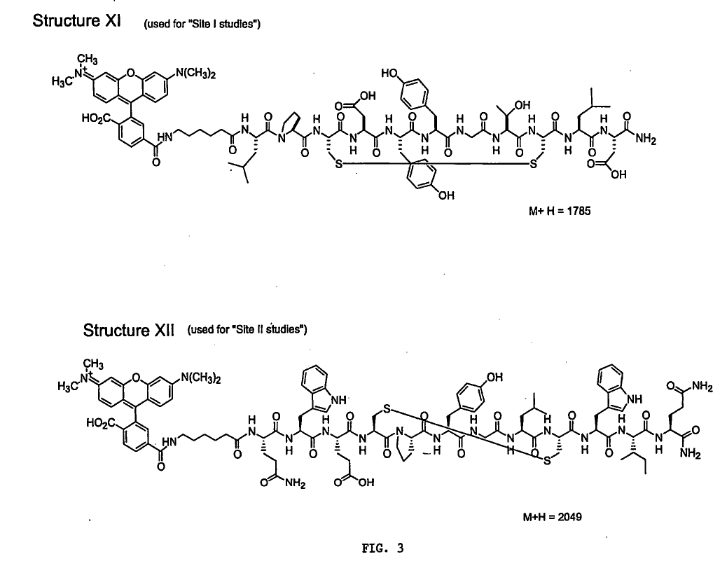

Site 2/fibrin: 0.1 mg/mL of fibrinogen was mixed with 0.6 μM of an optical agent (Structure XI, see FIG. 3) comprising tetramethylrhodamine as the optical dye. The mixture was coated (approximately 4-20 μL) onto a glass slide and cross-linking of fibrinogen was initiated with 1.3 μg/L of thrombin. Clotting occurred in approximately 15 sec. The slide was imaged using confocal fluorescence imaging (ex 555 nm, em 575 nm). Fibrin was detected based on the signal of the fibrin-optical agent complexes formed by the binding of Structure XI to fibrin on a lesion formed by cross-linking fibrinogen with thrombin.

Site 2/plasma clot: human plasma (platelet rich human plasma) was mixed with 0.6 μM of an optical agent (Structure XI, see FIG. 3) comprising tetramethylrhodamine as the optical dye. The mixture was coated onto a glass slide (approximately 4-20 μL) and clotting of plasma was initiated with 1.3 μg/L of thrombin. Clotting occurred within 15 sec. The slide was imaged using confocal fluorescence imaging (ex 555 nm, em 575 nm). Fibrin was detected based the signal of the fibrin-optical agent complexes formed by the binding of Structure XI to fibrin on a lesion (plasma clot) formed by clotting human plasma with thrombin.

Site 1/fibrin: 0.1 mg/mL of fibrinogen was mixed with 0.6 μM of an optical agent (Structure X) comprising tetramethylrhodamine as the optical dye. The mixture was coated (approximately 4-20 μL) onto a glass slide and cross-linking of fibrinogen was initiated with 1.3 μg/L of thrombin. Clotting occurred in about 15-20 seconds. The slide was imaged using confocal fluorescence imaging (ex 555 nm, em 575 nm). Fibrin is detected based on the signal of the fibrin-optical agent complexes formed by the binding of Structure X to fibrin on a lesion formed by cross-linking fibrinogen with thrombin.

In other examples, the optical agent was added in approximately stoichiometric amount to fibrinogen after the clotting of the fibrinogen had occurred on the surface of a slide. The optical agent was added after waiting a period of 10 times the clotting period (e.g., 150 sec.) by layering the solution over the clot on the slide and covering with a cover slip.

### Example 3

### Treatment of a Lesion

A guinea pig (Harley, male) is anaesthetized. An incision is made in the abdomen and the inferior vena cava (IVC) is isolated. The vessel is allowed to recover for 10 mins. A 1 cm portion of the IVC is clamped and human thrombin (50 μL, 4 units) is injected into the vessel to promote thrombus formation. The lower clamp is opened and closed to allow partial blood flow to the segment. After 2-3 mins., the clips are removed. The thrombus is allowed to age in the animal for 30 mins. At this point, the optical agent is administered at a dose of 0.02 μmol/kg, via injection into the jugular vein. After 30 mins., a catheter with an optical fluorescence detector is inserted into the IVC and the thrombus visualized by detecting the fluorescence signal emitted by the fibrin-optical agent complexes on the thrombus. Tissue plasminogen activator (tPA) is delivered through the catheter and the optical fluorescence signal decreases, indicating clot dissolution and lysis. TNKASE™ (Tenecteplase) is a commercially approved tissue plasminogen activator (tPA) produced by recombinant DNA technology and sold by Genetech. The drug is administered intravenously at a dose of 30-50 mg, depending on patient weight.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A method for treating an intravascular lesion in a patient comprising:
a) administering an optical agent, wherein the optical agent comprises a fibrin binding moiety and an optical dye, wherein the optical agent forms a fibrin-optical agent complex at the site of the lesion;
b) detecting a signal from the fibrin-optical agent complex using a device inserted near the lesion;
c) obtaining data about the lesion based on the signal from the fibrin-optical agent complex; and
d) delivering a therapy to at least a portion of the lesion based on the obtained data.

2. The method of claim 1, wherein the fibrin binding moiety comprises a peptide.

3. The method of claim 2, wherein the optical dye is covalently bound to the N-terminal amino acid of the peptide.

4. The method of claim 2, wherein the optical dye is covalently bound to the N-terminal amino acid of the peptide via a linker.

5. The method of claim 2, wherein the fibrin binding moiety comprises the amino acid sequence Cys-Asp-Tyr-Tyr-Gly-Thr-Cys (SEQ ID NO:1).

6. The method of claim 2, wherein the fibrin binding moiety comprises the amino acid sequence Cys-Pro-Tyr-Xaa-Leu-Cys (SEQ ID NO:2), wherein Xaa is Gly or Asp.

7. The method of claim 2, wherein the fibrin binding moiety comprises the amino acid sequence Cys-Hyp-Tyr(3×)-Xaa-Leu-Cys (SEQ ID NO:3), wherein 3× is selected from the group consisting of halogen, nitro-, and a trifluoromethyl group at the 3 position of the benzyl ring of the tyrosine, and wherein Xaa is Gly or Asp.

8. The method of claim 2, wherein the fibrin binding moiety comprises the amino acid sequence Phe-His-Cys-Hyp-Tyr(3-I)-Asp-Leu-Cys-His-Ile-Leu (SEQ ID NO:4).

9. The method of claim 3, wherein the N-terminal amino acid is selected from the group consisting of β-alanine, 6-aminohexanoic acid, and lysine.

10. The method of claim 2, wherein the optical dye is covalently bound to the C-terminal amino acid of the peptide.

11. The method of claim 2, wherein the optical dye is covalently bound to the C-terminal amino acid of the peptide via a linker.

12. The method of claim 3, wherein the C-terminus of the peptide is capped as a C-terminal amide.

13. The method of claim 3, wherein the C-terminus of the peptide is capped with a non-optical moiety.

14. The method of claim 3, wherein the C-terminal amino acid is in the D-configuration.

15. The method of claim 1, wherein the optical dye is selected from the group consisting of fluorescein, rhodamine, hematoporphyrin, fluoresdamine, indocyanine, tetramethylrhodamine, Cosin, erythrosine, coumarin, methyl-coumarins, pyrene, Malacite green, stilbene, Lucifer Yellow, Cascade Blue, Texas Red, and derivatives thereof.

16. The method of claim 1, wherein the optical agent is selected from the group consisting of:

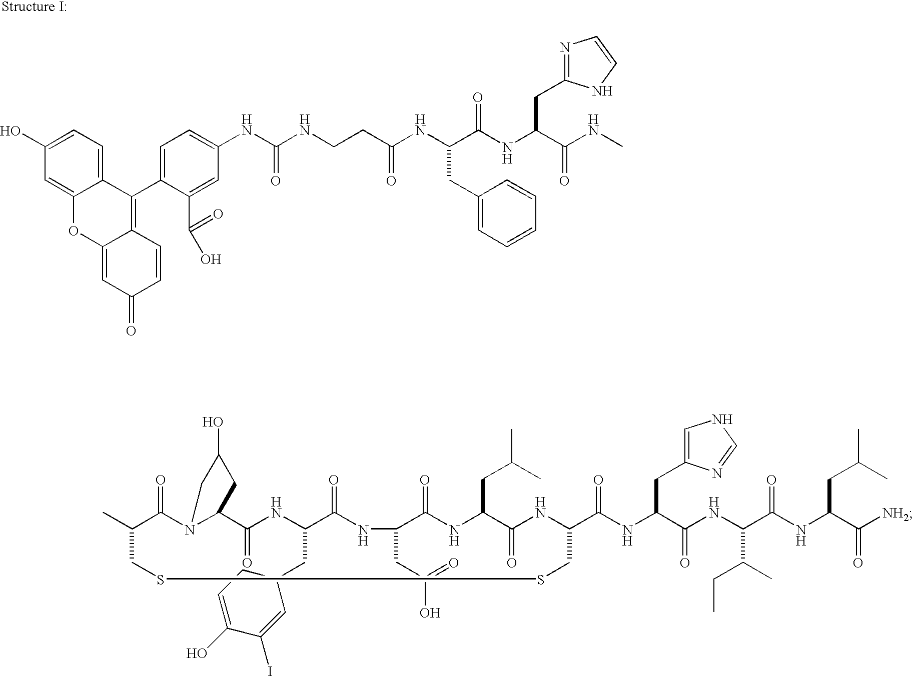

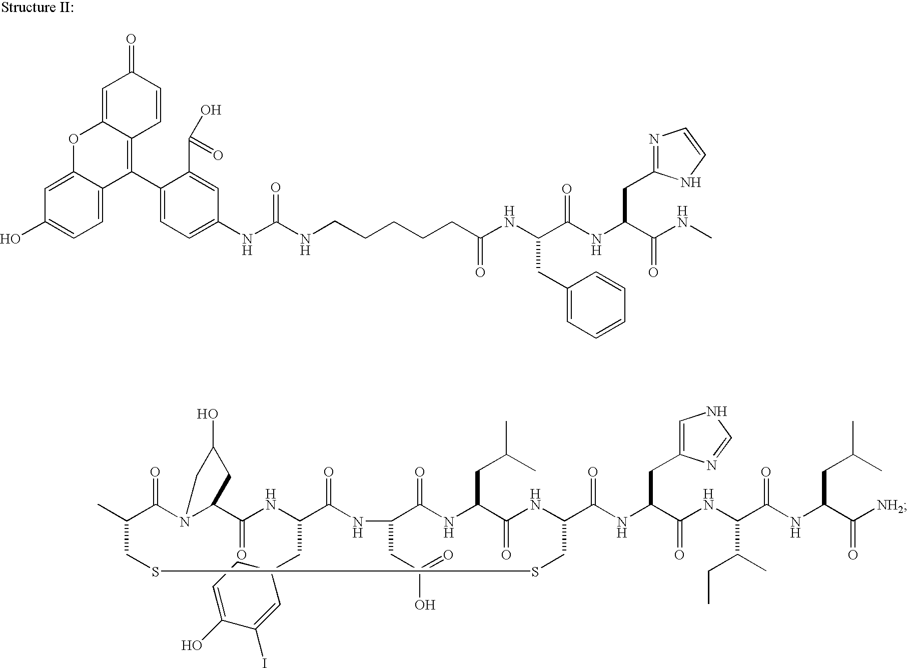

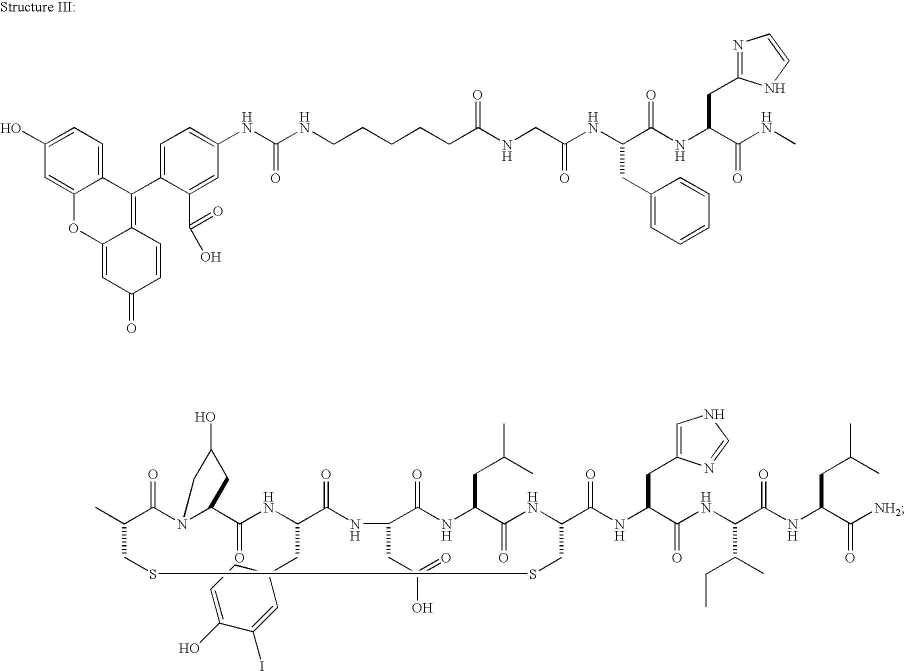

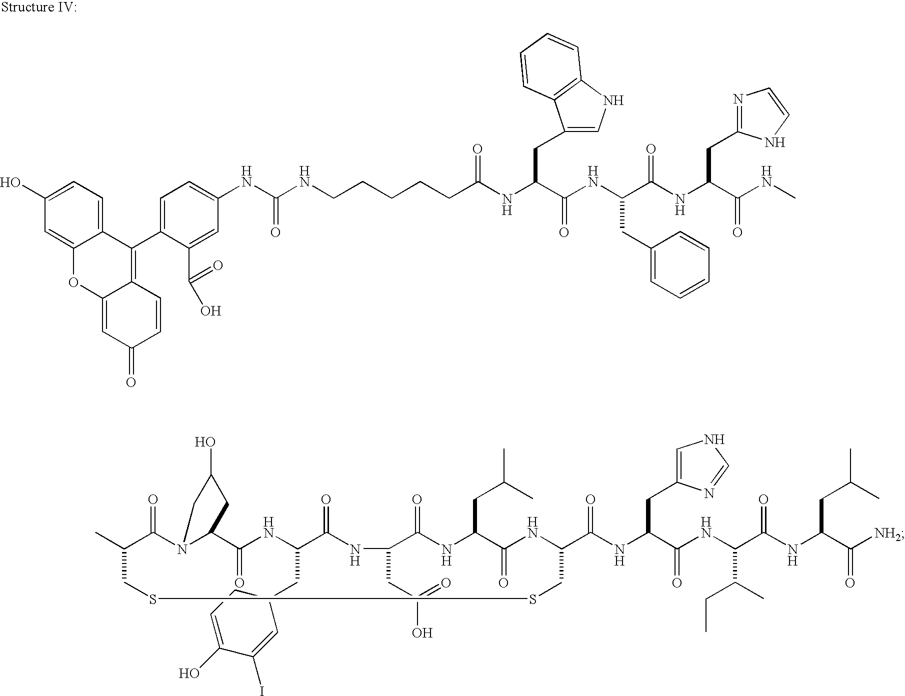

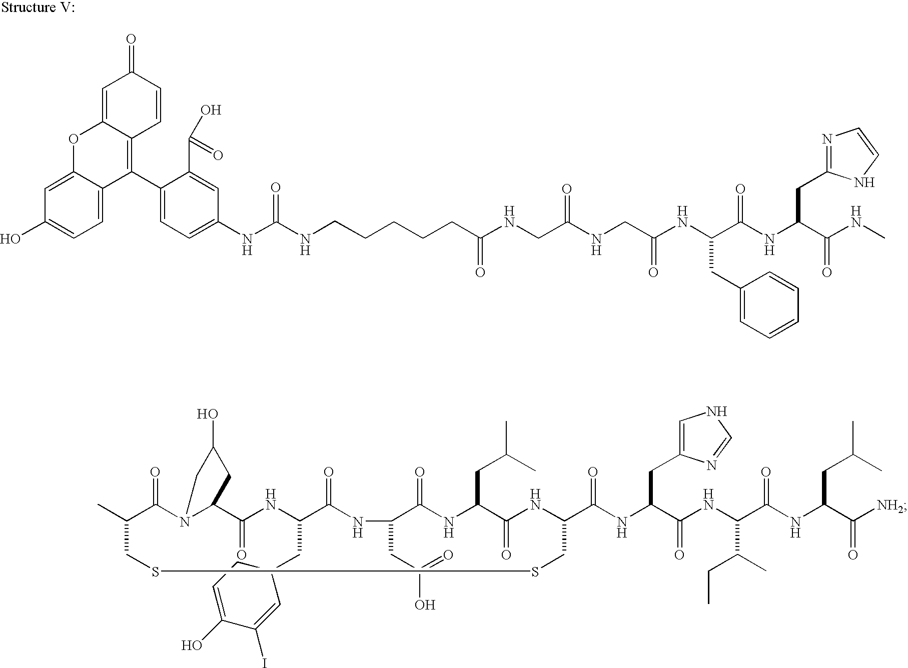

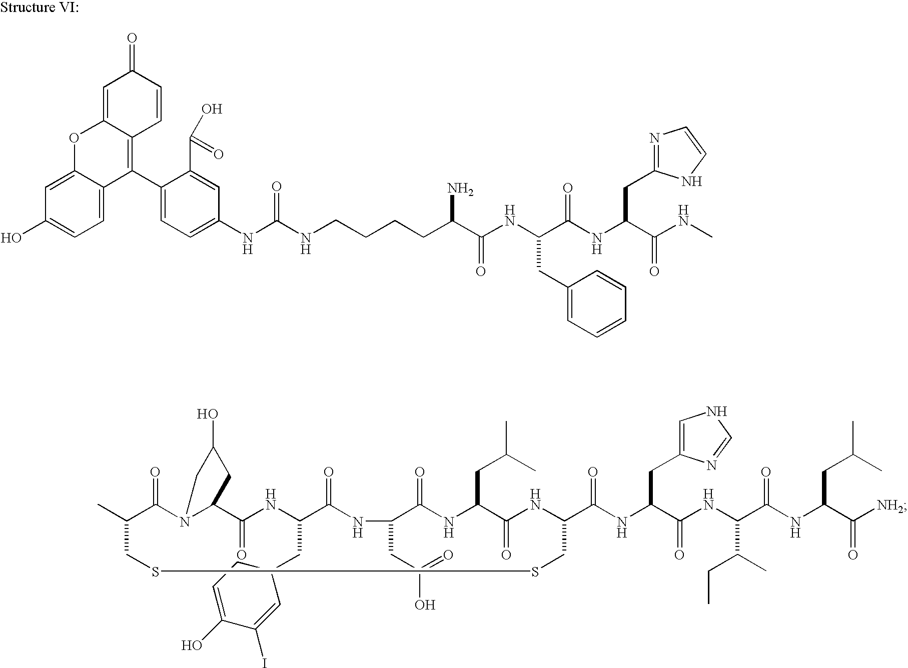

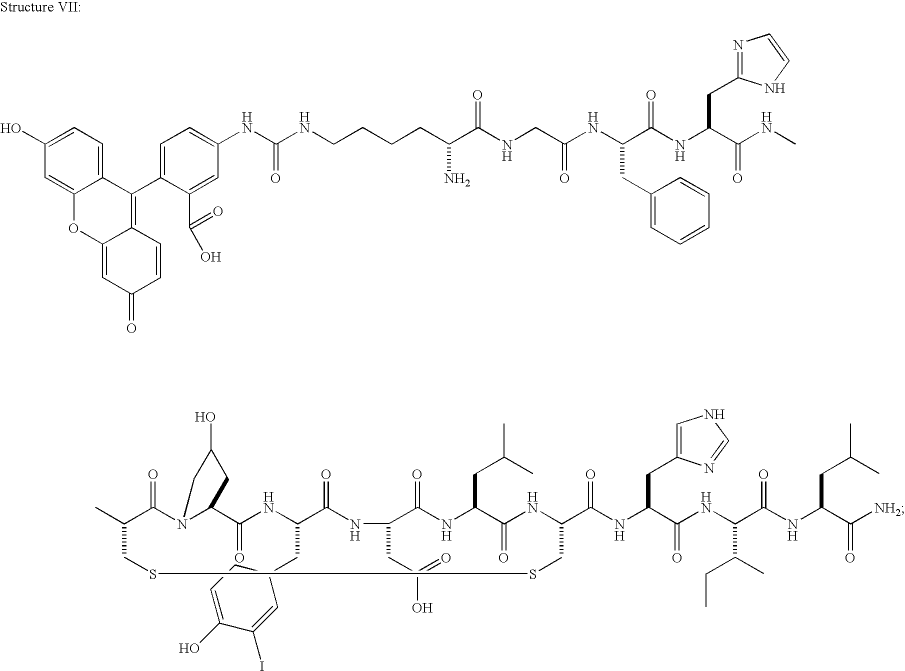

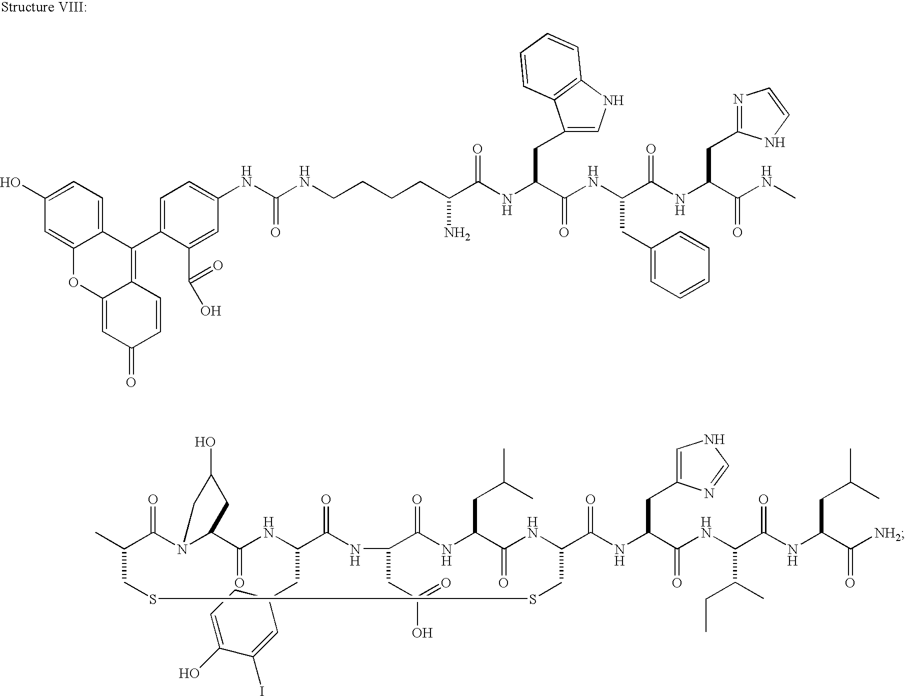

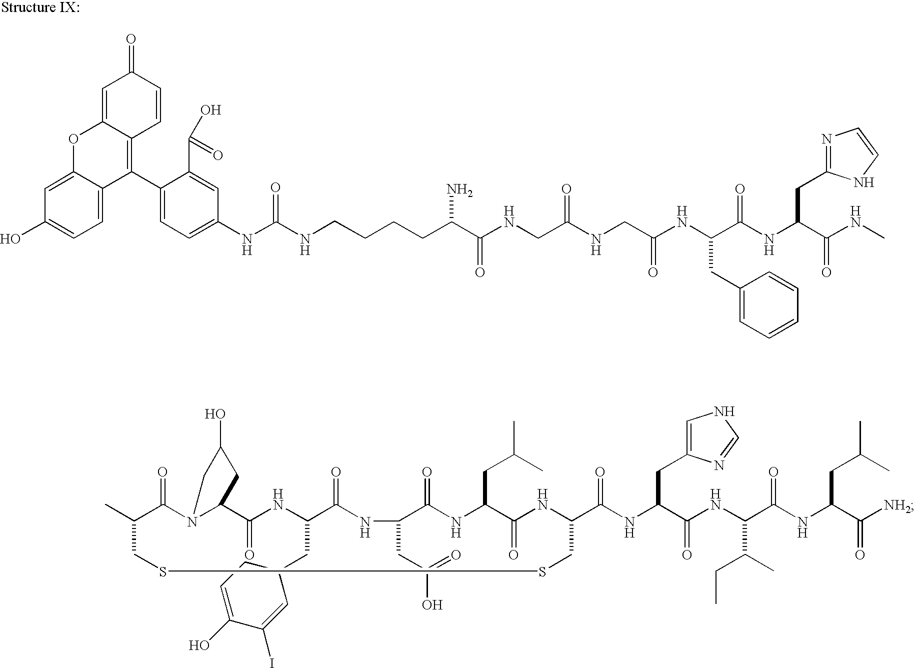

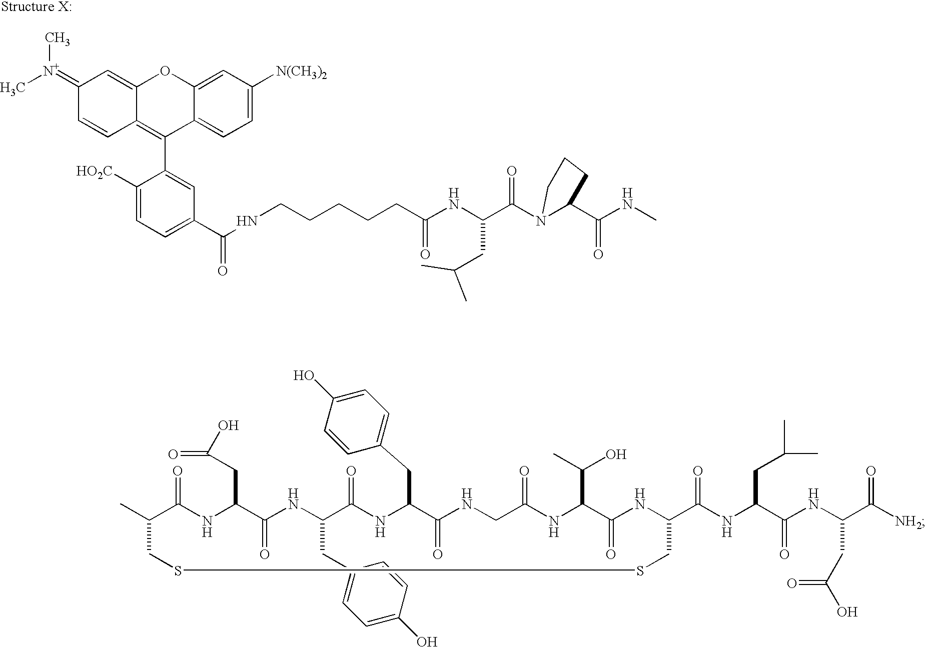

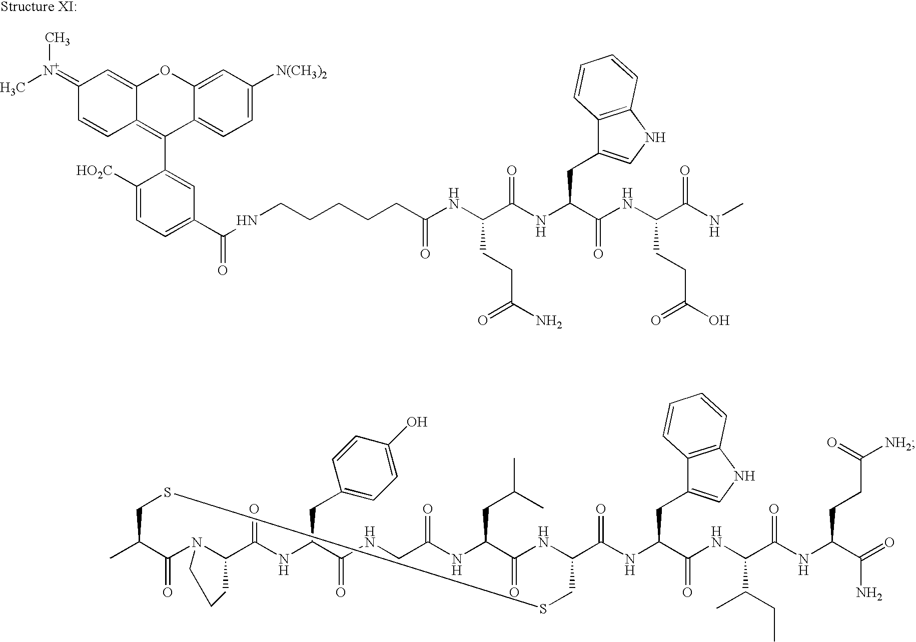

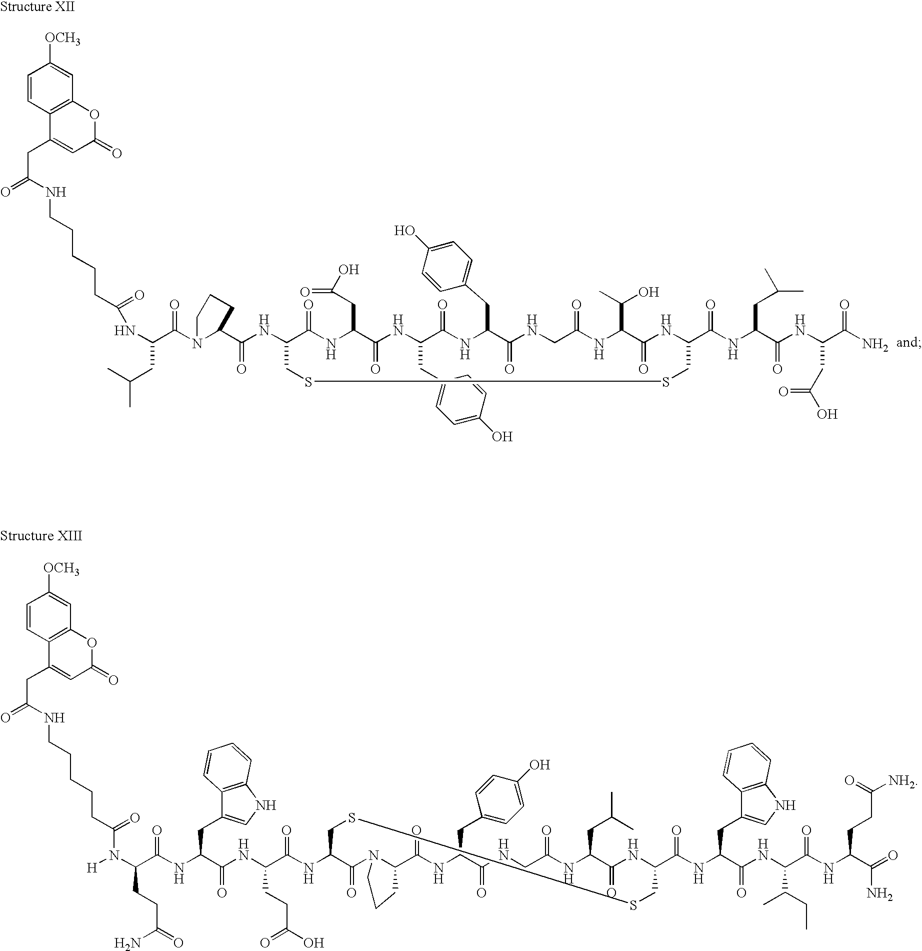

17. The method of claim 1, wherein the lesion is selected from the group consisting of a thrombus, a clot, an atherosclerotic plaque, and an embolus.

18. The method of claim 1, wherein the lesion comprises fibrin that is exposed to blood flowing in the blood vessel.

19. The method of claim 1, wherein the fibrin-optical agent complex has a dissociation constant value of less than about 10 μM.

20. The method of claim 1, wherein the fibrin-optical agent complex has a dissociation constant value of less than about 5 μM.

21. The method of claim 1, wherein the fibrin-optical agent complex has a dissociation constant value of less than about 1 μM.

22. The method of claim 1, wherein the fibrin-optical agent complex has a dissociation constant value of less than about 0.3 μM.

23. The method of claim 1, wherein the optical agent is administered orally or parenterally.

24. The method of claim 23, wherein the parenteral administration is intravenous, intraarterial, interstitial, intrathecal, subcutaneous, or intracavity administration.

25. The method of claim 1, wherein the device comprises a catheter and an optical detector.

26. The method of claim 25, wherein the optical detector is a fluorescence emission detector.

27. The method of claim 25, wherein the device further comprises an excitation source.

28. The method of claim 25, wherein the device is inserted near the lesion in a cavity, a tissue, an interstitial space, or a blood vessel.

29. The method of claim 25, wherein the device is inserted in the same blood vessel as the lesion.

30. The method of claim 1, wherein the device is capable of delivering the therapy to at least a portion of the lesion.

31. The method of claim 1, wherein the therapy comprises a thrombolytic agent.

32. The method of claim 31, wherein the thrombolytic agent is selected from the group consisting of tissue plasminogen activator, streptokinase, antistreplase, and urokinase.

33. The method of claim 31, wherein the thrombolytic agent is administered intravenously at a site remote from the lesion.

34. The method of claim 31, wherein the thrombolytic agent is delivered to at least about 90% of the surface of the lesion.

35. The method of claim 31, wherein the thrombolytic agent is delivered to at least about 50% of the surface of the lesion.

36. The method of claim 31, wherein the thrombolytic agent is delivered to about 10% of the surface of the lesion.

37. The method of claim 1, wherein the therapy comprises mechanical manipulation of the lesion.

38. The method of claim 37, wherein the mechanical manipulation is selected from the group consisting of balloon angioplasty and laser ablation of the lesion.

39. The method of claim 1, further comprising e) detecting the signal from the fibrin-optical agent complex during the delivery of the therapy.

40. The method of claim 39, further comprising f) stopping the delivery of the therapy when the signal of the fibrin-optical agent complex decreases to a predetermined value.

41. The method of claim 40, wherein the therapy is stopped when the signal of the fibrin-optical agent complex is less than about 90% of the signal before delivery of the therapy.

42. The method of claim 40, wherein the therapy is stopped when the signal of the fibrin-optical agent complex is less than about 50% of the signal before delivery of the therapy.

43. The method of claim 40, wherein the therapy is stopped when the signal of the fibrin-optical agent complex is less than about 10% of the signal before delivery of the therapy.

44. The method of claim 10, wherein the N-terminus of the peptide is alkylated.

45. The method of claim 10, wherein the N-terminal amino acid is in the D-configuration.

46. A composition comprising an optical agent, wherein the optical agent comprises an optical dye covalently linked to the N-terminus of a peptide fibrin binding moiety (FBM) via a linker, said optical agent having the general formula:

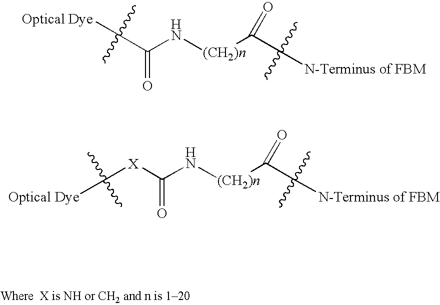

47. The composition of claim 46, wherein the optical agent is selected from the group consisting of: 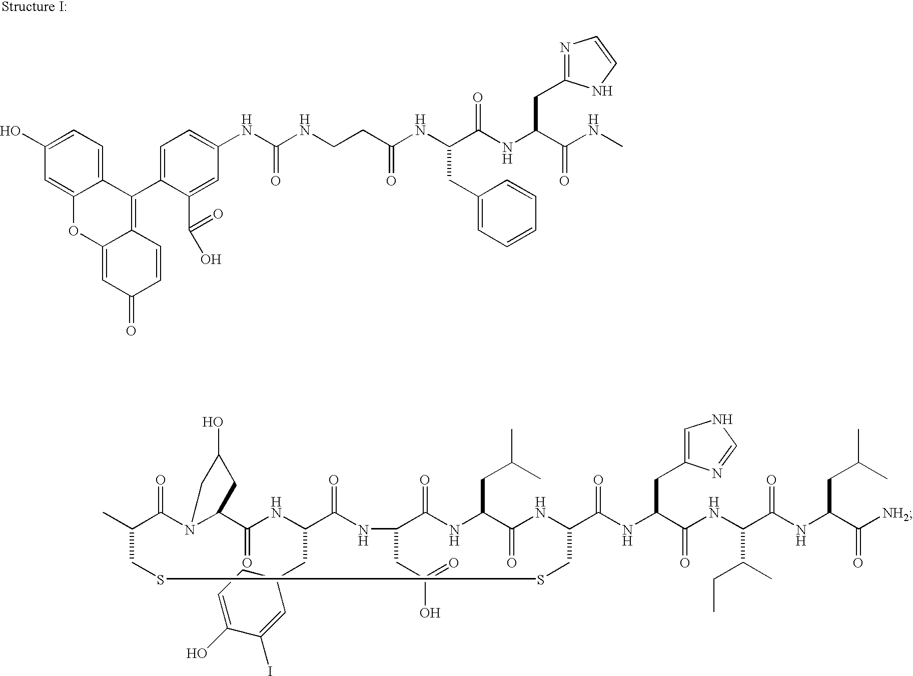 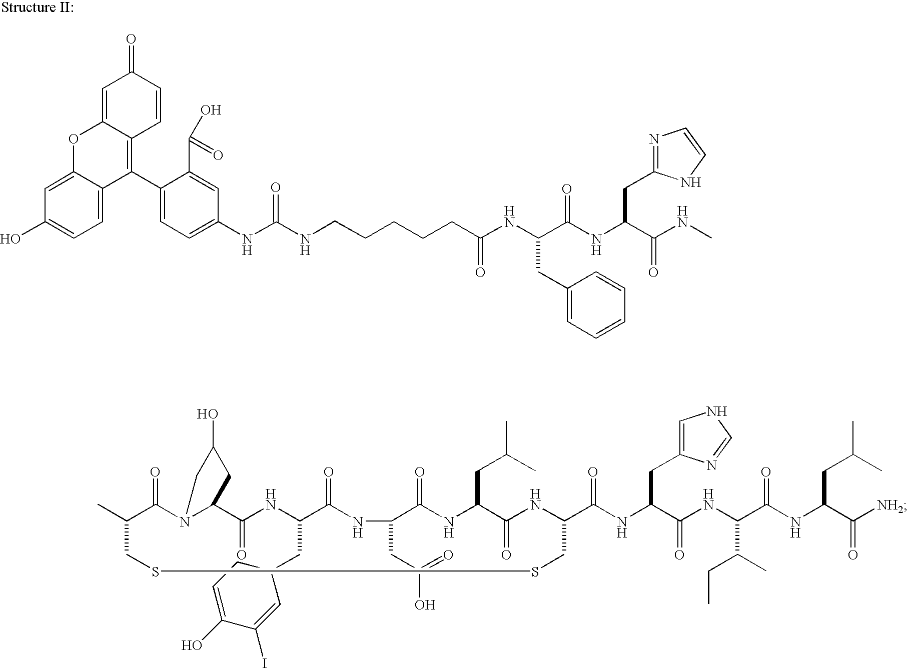 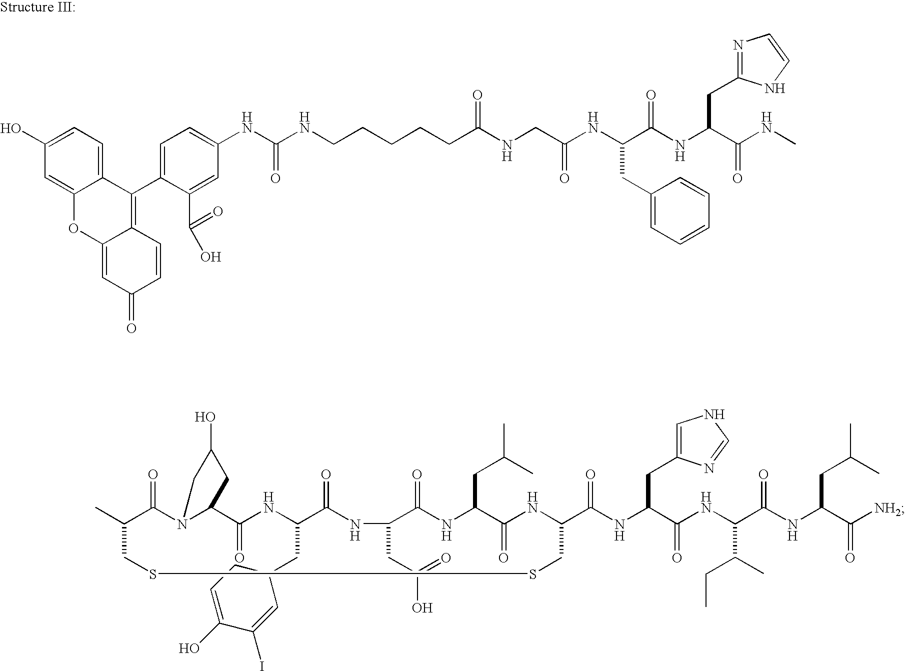 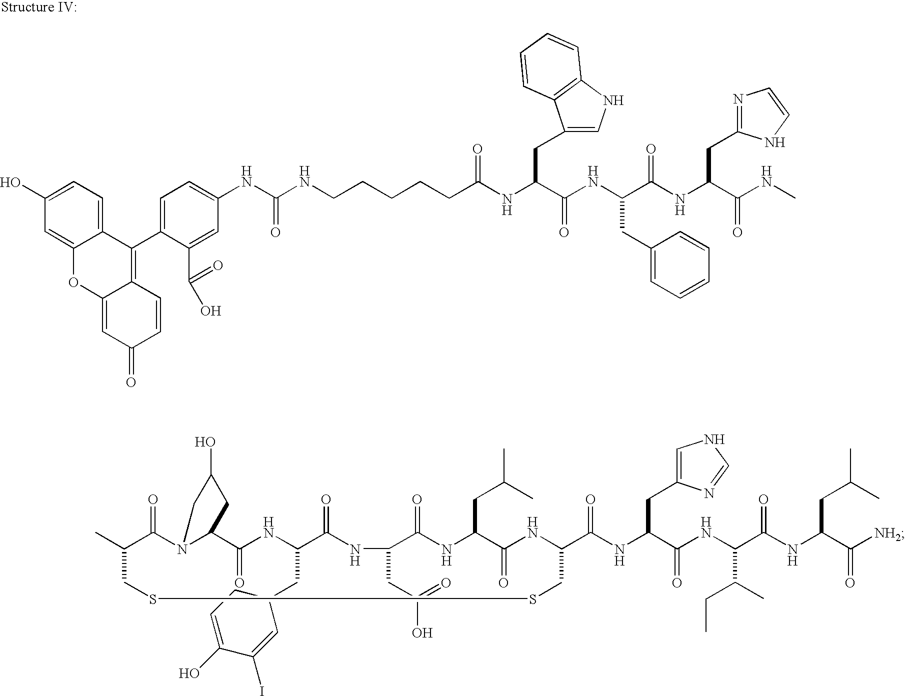 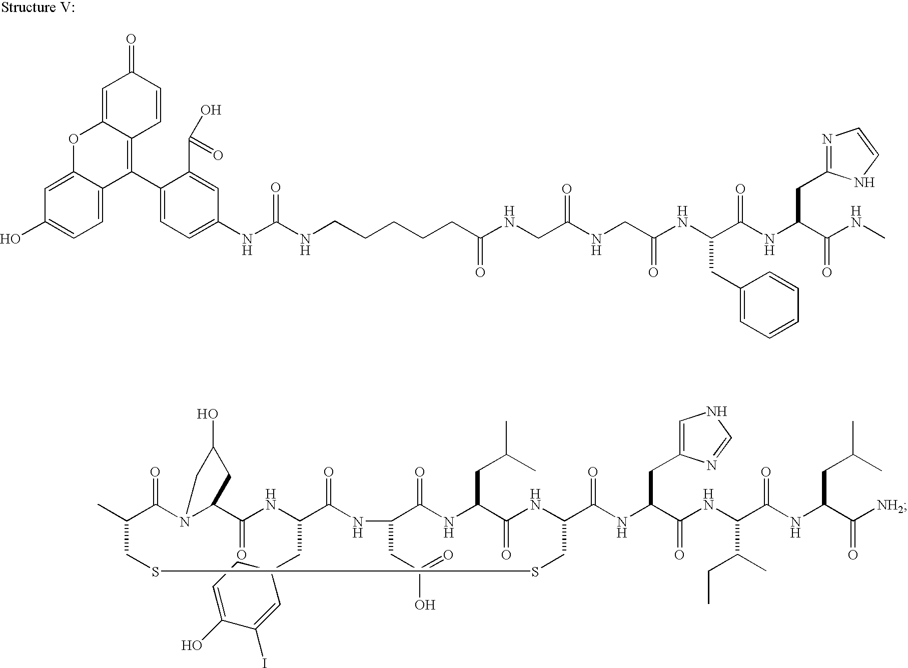 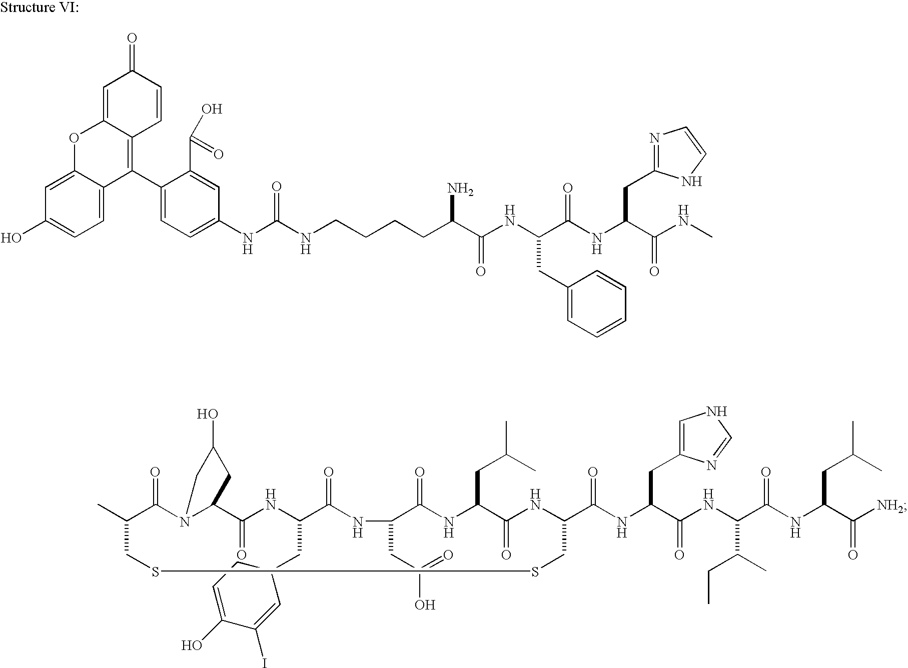 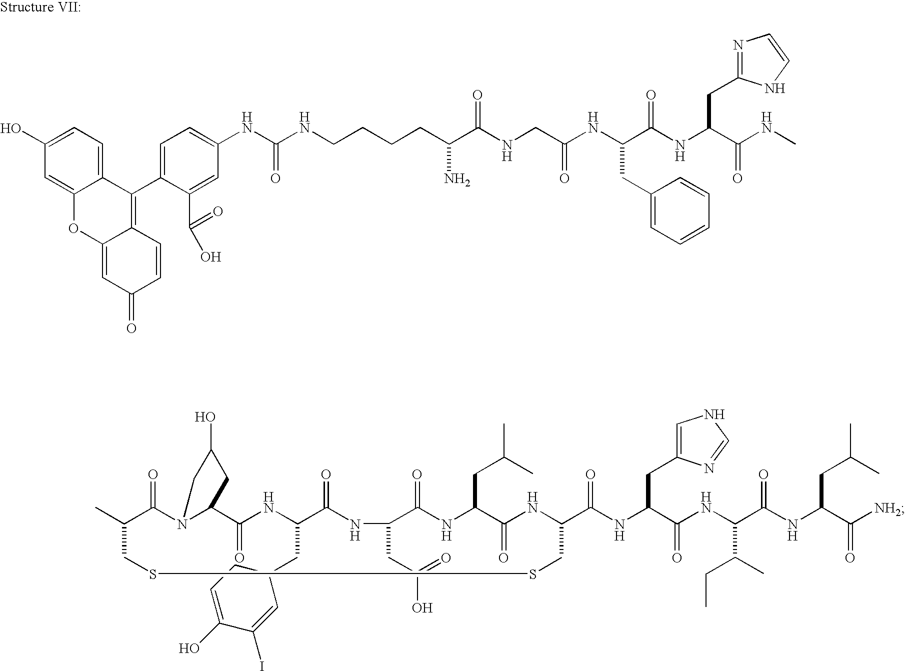 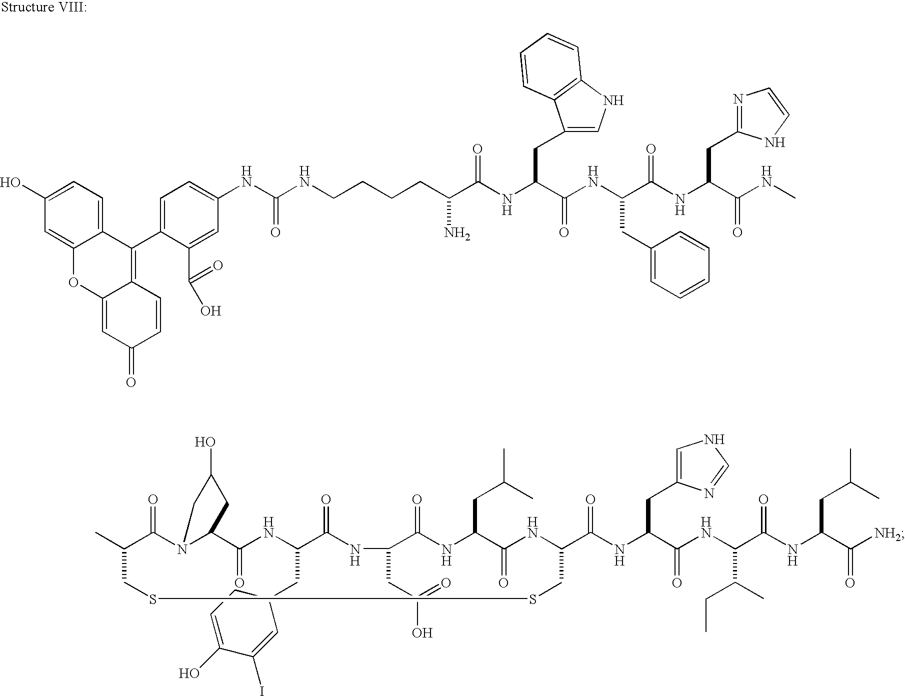 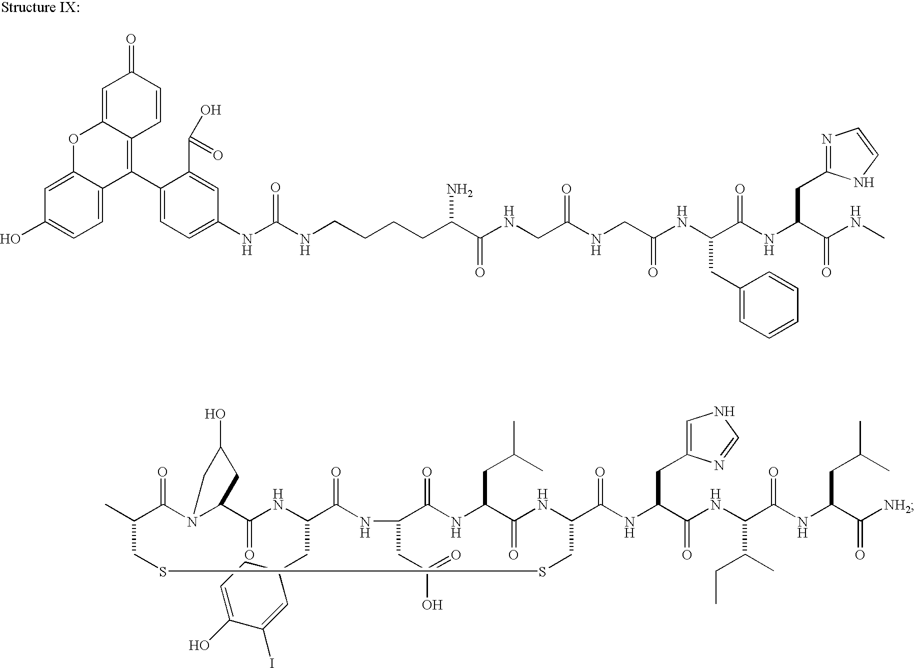 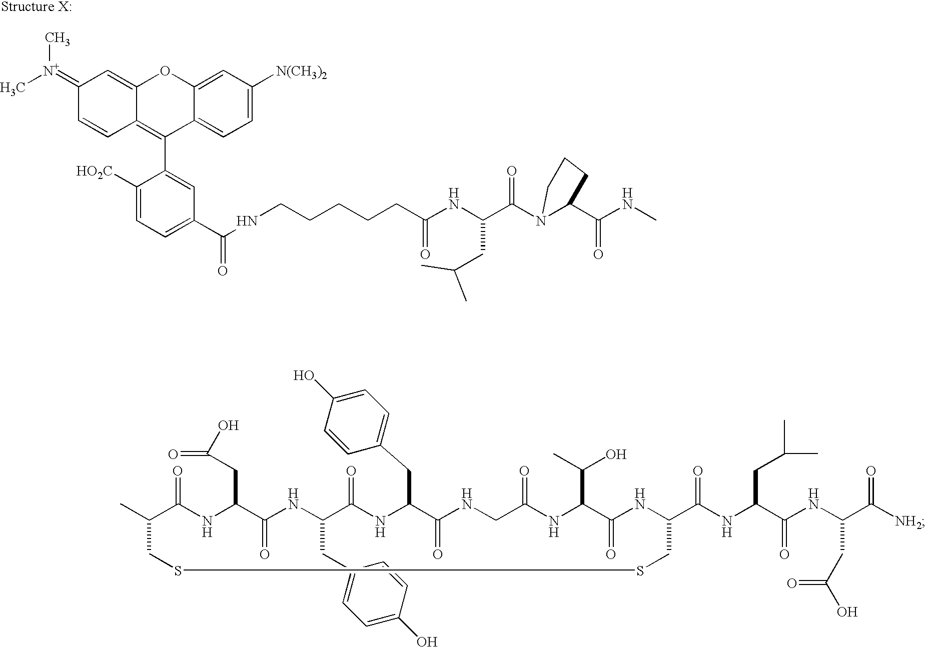 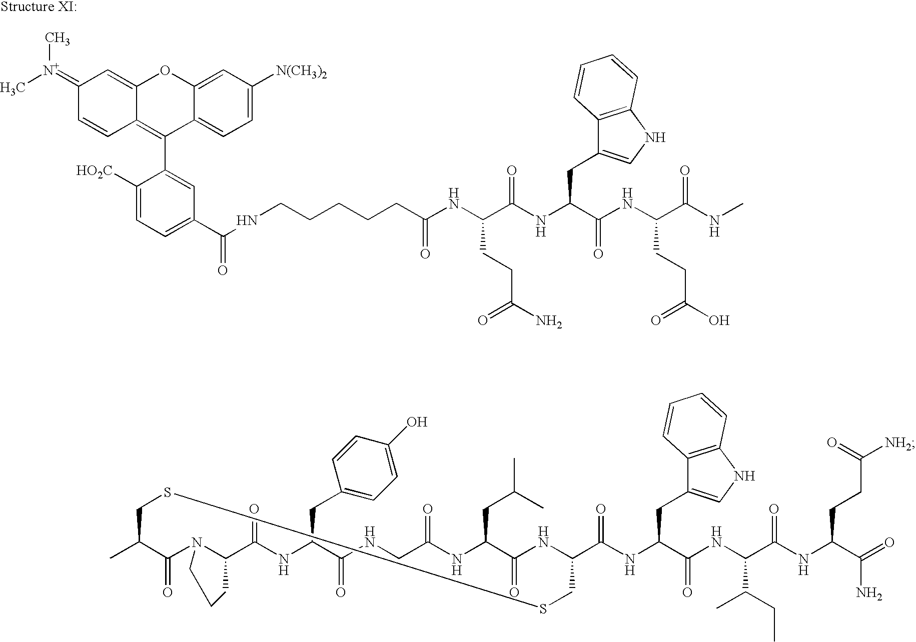 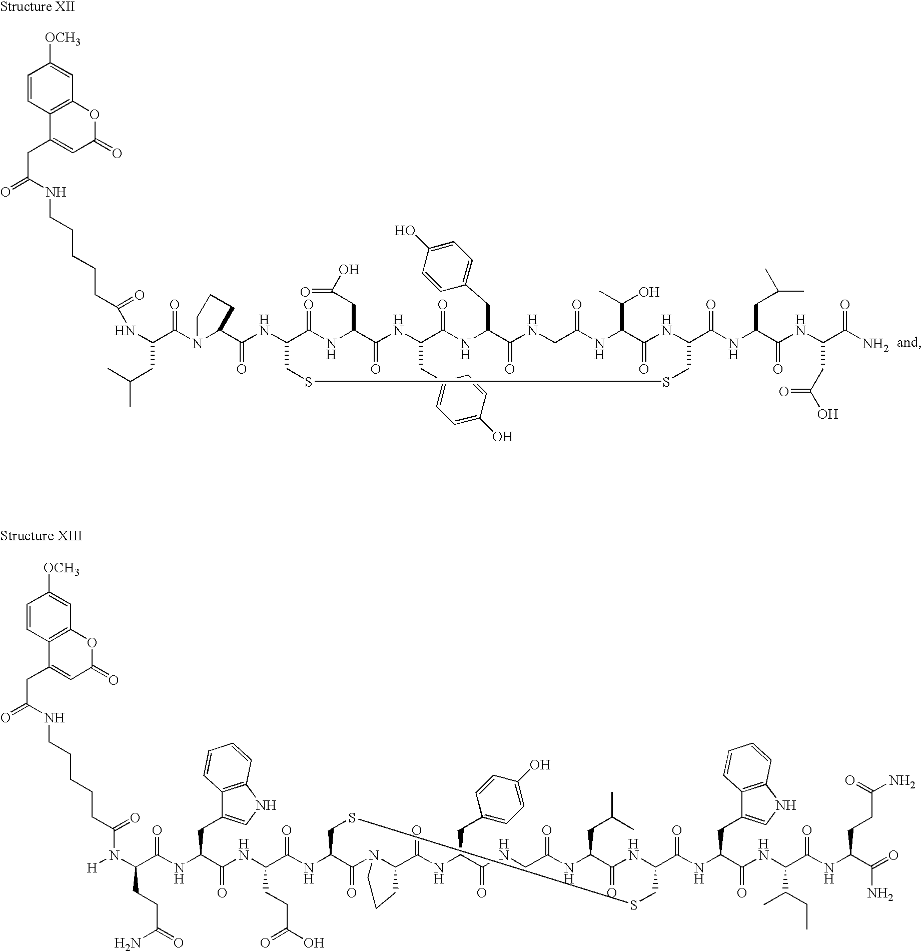
and a pharmaceutically acceptable salt thereof.

48. A formulation comprising the composition of claim 47, wherein the formulation comprises at least one ingredient selected from the group consisting of solubilizing agents, excipients, carriers, adjuvants, vehicles, preservatives, a local anesthetic, flavorings, and colorings

49. A kit comprising the composition of claim 47.

50. A method to treat a thrombus in a blood vessel in a patient, said method comprising:
a) administering an optical agent, said agent having the structure: 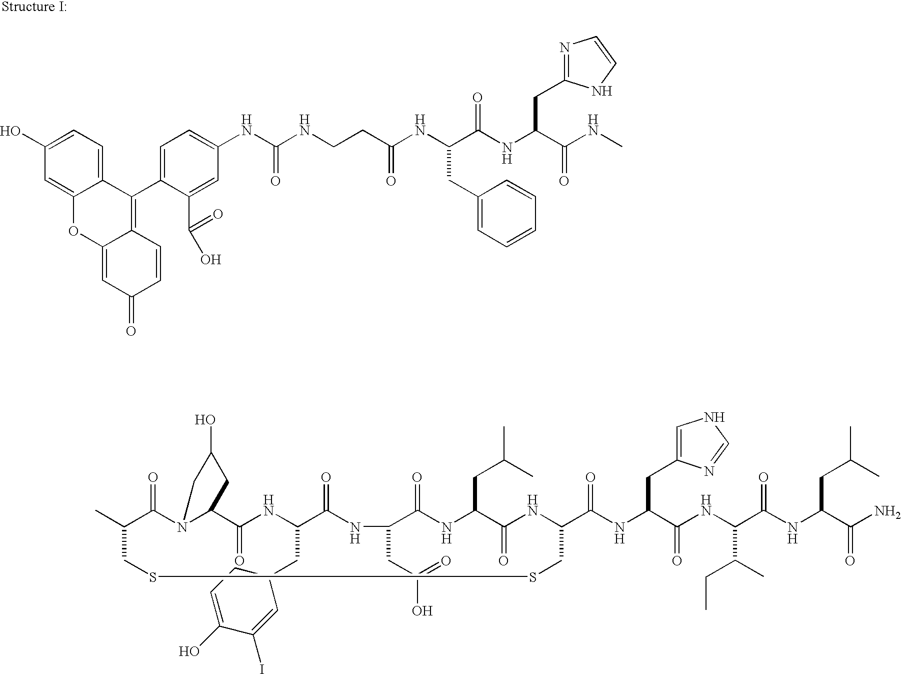
to form a fibrin-optical agent complex;
b) inserting a catheter in the blood vessel having said thrombus to obtain information about said thrombus, said information based on detecting a fluorescence emission signal of said fibrin-optical agent complex; and
c) delivering with said catheter a thrombolytic therapy comprising tissue plasminogen activator (tPA) based on said information to about 90% of said thrombus so that the size of said thrombus is reduced.
